(19) United States
(12) Patent Application Publication
(10) Pub. No.: US 2023/0177682 A1
(43) Pub. Date: Jun. 8, 2023

(54) SYSTEMS AND METHODS FOR CHARACTERIZING A TUMOR MICROENVIRONMENT USING PATHOLOGICAL IMAGES

(71) Applicant: The Board of Regents of The University of Texas System, Austin, TX (US)

(72) Inventors: Guanghua Xiao, Dallas, TX (US); Yang Xie, Dallas, TX (US); Ruichen Rong, Dallas, TX (US); Shidan Wang, Dallas, TX (US)

(73) Assignee: The Board of Regents of The University of Texas System, Austin, TX (US)

(21) Appl. No.: 17/998,037

(22) PCT Filed: May 6, 2021
(86) PCT No.: PCT/US21/31160

§ 371 Date: Nov. 6, 2022

Related U.S. Application Data
(60) Provisional application No. 63/021,068, filed on May 6, 2020.

(51) Int. Cl. G06T 7/00; G06V 10/82; G06V 20/69; G06T 7/11; G06V 10/44; G06V 10/764; G16H 50/20; G16H 50/30

(52) U.S. Cl. CPC G06T 7/0012; G06V 10/82; G06V 20/698; G06T 7/11; G06V 10/454; G06V 10/764; G16H 50/20; G16H 50/30; G06T 2207/30024; G06T 2207/30096; G06T 2207/20084

(57) ABSTRACT

Implementations discussed and claimed herein provide systems and methods for characterizing patient tissue of a patient. In one implementation, a pathological image of the patient tissue is received. Nuclei of the plurality of cells in the pathological image are simultaneously segmented and classified using a histology-based digital staining system. The nuclei of the plurality of cells are segmented according to spatial location and classified according to cell type, thereby generating one or more groups of nuclei. Each of the one or more groups of nuclei have an identified cell type. A composition and a spatial organization of a tumor microenvironment of the patient tissue is determined based on the one or more groups of nuclei. A prognostic model for the patient is generated based on the composition and the spatial organization of the tumor microenvironment.

### CROSS-REFERENCE TO RELATED APPLICATIONS

This application is a national stage application filed under 35 U.S.C. § 371 of International Patent Application No. PCT/US2021/031160 entitled “Systems and Methods for Characterizing a Tumor Microenvironment using Pathological Images” filed May 6, 2021, which claims the benefit of U.S. Provisional Patent Application No. 63/021,068 entitled “Systems and Methods for Characterizing a Tumor Microenvironment using Pathological Images” filed May 6, 2020, the contents of which are incorporated by reference in their entireties.

### BACKGROUND

### 1. Field

The present disclosure is directed to systems and methods for characterizing patient tissue through a quantification of a tumor microenvironment from a pathological image and more particularly to a Histology-Based Digital Staining system that identifies cells with increased accuracy in the tumor microenvironment using deep-learning and generates a prognostic model to predict patient prognosis and optimize patient treatment.

### 2. Discussion of Related Art

Cancer is often diagnosed based on an examination of slides of tissue samples of a patient. Hematoxylin and Eosin (H&E) stained tissue slide scanning facilitates such examination by producing pathology images that capture histological details of the patient tissue in high resolution. However, analyzing these high resolution images to understand the tumor microenvironment (TME) of the patient tissue to make clinical determinations is conventionally impractical. More particularly, to understand the TME, the millions of cells included in a slide image are manually labeled by an expert pathologist, resulting in significant resources being expended and valuable time being lost. Additionally, in pathology image analysis, three-dimensional tissue structures are captured as two-dimensional images, such that the cells may appear to touch or overlap with each other in the pathology images. As such conventional attempts to automatically identify cells through image segmentation techniques are often incomplete or inaccurate, among other concerns. It is with these observations in mind, among others, that various aspects of the present disclosure were conceived and developed.

### BRIEF DESCRIPTION OF THE DRAWINGS

In order to describe the manner in which the advantages and features of the disclosure can be obtained, reference is made to implementations thereof which are illustrated in the appended drawings. One of skill in the art will understand that the reference numbers in the following figures are repeated throughout FIGS. 1-10 so as to refer to the same or substantially the same features. Understanding that these drawings depict only exemplary implementations of the disclosure and are not therefore to be considered to be limiting of its scope, the principles herein are described and explained with additional specificity and detail through the use of the accompanying drawings.

FIG. 1 is a block diagram showing an example system for characterizing patient tissue, including a tumor microenvironment.

FIG. 2 illustrates segmentation of cell nuclei following an extraction of an image patch from a region of interest of an example pathology image.

FIG. 3 shows an example pathology image of patient tissue and a characterized tumor microenvironment including a composition and a spatial organization of the patient tissue following cell nuclei segmentation and classification.

FIG. 4 depicts segmentation and classification of cell nuclei for an example pathology image using a mask region-based convolutional neural network.

FIG. 5 illustrates an extraction of topological features from an example nuclei spatial organization to characterize the tumor microenvironment.

FIG. 6 shows an example plot of predicted high-risk and low-risk groups generated based on a pathology model.

FIG. 7 shows diagrams illustrating example edge-conditioned (EC) convolution.

FIG. 8 is a diagram showing an edge angle between a first nucleus and a second nucleus.

FIG. 9 illustrates an example graph convolutional network (GCN) to classify adenocarcinoma (ADC) versus squamous carcinoma (SCC).

FIG. 10 illustrates an example performance of GCN in histology subtype classification.

FIG. 11 shows an example visual contribution of each tumor cell nuclei to a final histological classification.

FIG. 12 depicts example visualization of GCN plotted for an ADC image patch.

FIG. 13 shows example visualization of GCN plotted for an SCC image patch.

FIG. 14 shows an example predictive value of GCN in target therapy response prediction.

FIG. 15 illustrates an example whole-slide GCN prediction.

FIG. 16 depicts a comparison of example image features between ADC and SCC.

FIG. 17 illustrates an example receptive field of a single node in a EC convolutional layer.

FIG. 18 illustrates an example image restoration architecture.

FIG. 19 shows example images enhanced using the image restoration architecture.

FIG. 20 shows example images enhanced and normalized using the image restoration architecture.

FIG. 21 illustrates example changes to images using a fine-tuned mask model of the image restoration architecture.

FIG. 22 depicts an example of whole-slide histology-based digital staining.

FIG. 23 illustrates an example automatic tumor region detection.

FIG. 24 shows an example visualization of EGFR TKI response prediction model.

FIG. 25 shows an example validation of image feature-based epidermal growth factor (EGFR) tyrosine kinase inhibitors (TKI) response prediction model along with example predictive value of image feature-based EGFR TKI response prediction model.

FIG. 26 illustrates example pathology images of predicted TKI responders.

FIG. 27 illustrates example pathology images of predicted TKI non-responders.

FIG. 28 shows plots of example gene set enrichment analysis results correlating mRNA expression level with tumor-tumor interaction.

FIG. 29 shows an example of gene set enrichment analysis (GSEA) results correlating mRNA expression with tumor-tumor interaction and tumor-stroma interaction.

FIG. 30 shows example gene expression heatmaps from which a relationship between tumor-stroma interaction and mRNA expression of intersecting genes can be understood.

FIG. 31 illustrates genes involved in MET and EGFR mediated PIP3 activation pathways.

FIG. 32 illustrates an example comparison among patient groups stratified by predicted responding group, EGFR TKI treatment, and EGFR mutation type.

FIG. 33 illustrates example operations for characterizing patient tissue of a patient.

FIG. 34 is an example network environment that may implement aspects of the presently disclosed technology

FIG. 35 is a functional block diagram of an electronic device including operational units arranged to perform various operations of the presently disclosed technology.

FIG. 36 is an example computing system that may implement various systems and methods discussed herein.

### DETAILED DESCRIPTION

It will be appreciated that numerous specific details are set forth in order to provide a thorough understanding of the implementations described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the implementations described herein.

Aspects of the present disclosure generally involve a histology-based digital staining system that utilizes artificial intelligence to digitally stain pathology images and characterize a tumor microenvironment (TME) and predict patient clinical outcomes. In one aspect, the histology-based digital staining system applies a learned mask region-based convolutional neural network (Mask R-CNN) to simultaneously segment and classify nuclei of cells in a pathology image of patient tissue. The pathology image may be a slide image of the patient tissue or a patch of a region of interest from the slide image. A characterized tumor microenvironment, including a composition and a spatial organization of the cells of the patient tissue, is generated following the nuclei segmentation and classification, from which a prognostic model for the patient is generated to understand patient survival outcomes and clinical treatment outcomes. Additionally, a correlation with gene expression of biological pathways is determined based on the composition and the spatial organization of the cells.

The presently disclosed technology thus generally dissects the TME from pathology images for a patient and uses the spatial organization of different cell types to predict patient survival and determine associations with gene expression of biological pathways. Accordingly, the presently disclosed technology assists pathologists in the diagnosis of different types of cancer and lymph node metastasis, as well as quantitatively characterizes the spatial distribution of tumor-infiltrating lymphocytes, thereby predicting patient response to immunotherapy. Further, in connection with clinical practice, the presently discloses technology: reduces pathologist time in analyzing pathology images to identify what may be small tumor cells, thereby expediting diagnosis and treatment; optimizes treatment for individual patients based on predicted patient prognosis; and anticipates treatment outcomes, including patient response to immunotherapy based on the spatial distribution of lymphocytes and their interaction with the tumor region, thereby further optimizing patient treatment.

The various systems and methods disclosed herein generally provide for characterization of a tumor microenvironment from pathology images for a patient using a histology-based digital staining system. The example implementations discussed herein reference lung tissue and certain cell types and cancers. However, it will be appreciated by those skilled in the art that the presently disclosed technology is applicable to other types of tissue, cell types, and cancers.

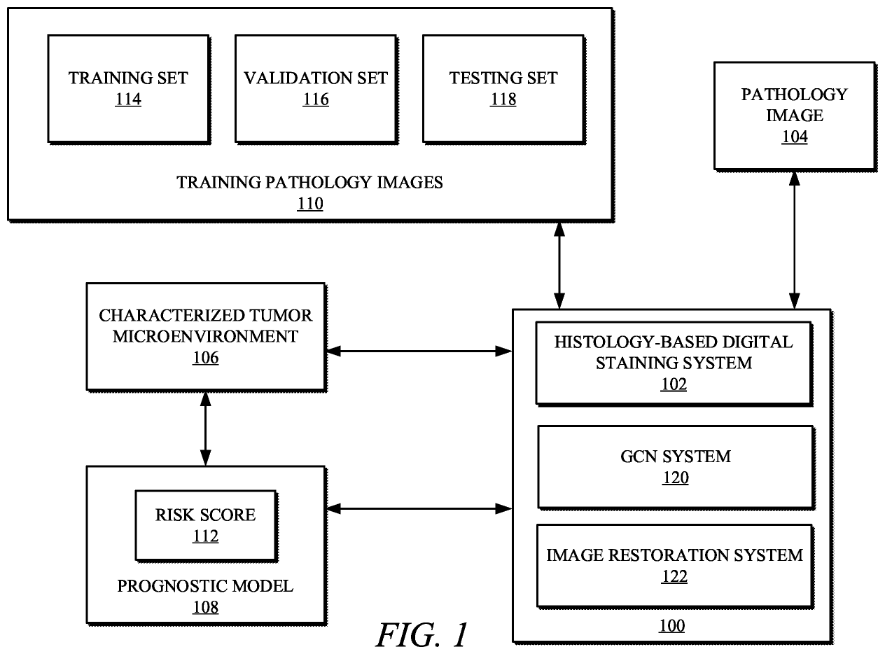

To begin a detailed description of an example system 100 for characterizing patient tissue, including a tumor microenvironment, reference is made to FIG. 1. In one implementation, the system 100 includes a histology-based digital staining system 102, a GCN system 120, and an image restoration system 122. The histology-based digital staining system 102 is configured to receive one or more pathology images 104. The pathology images 104 may be captured using Hematoxylin and Eosin (H&E) stained tissue slide scanning apparatuses or similar scanners, imagers, and/or kits. As such, the pathology images 104 may include H&E pathology images, as well as other pathology images of patient tissue.

Each pathology image 104 includes histological details in high resolution of patient tissue for a patient. The patient tissue includes a plurality of different types of cells. In the context of tissue analysis for cancer diagnosis or treatment, the pathology images 104 include information for tumor grade and subtype classification, as well as regarding the TME, such as the spatial organization of different types of cells in the patient tissue.

Cell spatial organization reveals cell growth patterns and the spatial interactions among different types of cells, which provides insight into tumor progression and metastasis. For example, spatial organization and architecture of tumor infiltrating lymphocytes (TIL) may impact the TME, alone or in connection with the interactions among different types of cells.

The major cell types in a malignant tissue include tumor cells, stromal cells, lymphocytes, and macrophages. Stromal cells are mainly connective tissue cells, such as fibroblasts and pericytes. The interactions between tumor cells and stromal cells may significantly impact cancer progression and metastasis inhibition. TIL are mainly white blood cells that have migrated into a tumor region. They are a mix of different types of cells, in which T cells are the most abundant population. The spatial organization of TIL has been associated with patient outcome and molecular profiles in multiple tumor types. Macrophages are inflammatory cells, and inflammation in tumor niches may be a prognostic marker and correlated with tumor progression. Other tissues and cellular structures existing in the TME include blood vessels and necrosis.

In one implementation, the histology-based digital staining system 102 examines the pathology image 104 to automatically segment and classify different types of cell nuclei. Cell boundaries of tumor cells and stromal cells are often unclear in H&E stained pathology images. Accordingly, the histology-based digital staining system 102 segments and classifies cell nuclei instead of whole cells. Moreover, the histology-based digital staining system 102 segments red blood cells and karyorrhexis to represent blood vessels and necrosis, respectively, to quantify blood vessels and necrosis and characterize their interactions with tumor cells, stromal cells, lymphocytes and macrophages.

Generally, the histology-based digital staining system 102 computationally stains different types of cell nuclei to facilitate examination of tissue images in connection with diagnosis and treatment optimization, as well as to characterize and study the TME. Stated differently, the histology-based digital staining system 102 generates a characterized TME 106, which may be utilized in generating a prognostic model 108.

In one implementation, the histology-based digital staining system 102 utilizes a deep learning architecture to simultaneously segment and classify the cell nuclei. For example, the histology-based digital staining system 102 may utilize a Mask R-CNN architecture. During segmentation and classification, the histology-based digital staining system 102 generates bounding boxes and segmentation masks for each instance of cell nuclei in the pathology image 104. More particularly, in one example, the histology-based digital staining system 102 generates positive and negative anchors along with anchor box refinement in the pathology image 104, providing anchor sorting and filtering of detection boxes. The bounding boxes are refined in a second stage in connection with the cell nuclei in the pathology image 104. The histology-based digital staining system 102 generates masks, which are scaled and placed on the pathology image 104 relative to the bounding boxes. Using the region-based approach, the histology-based digital staining system 102 segments the cell nuclei according to their spatial location in the pathology image 104 and classifies the cell type for each of the nuclei.

Using a Mask R-CNN architecture, the histology-based digital staining system 102 detects the particular shape, spatial location, and cell type of each nucleus in the pathology image 104. In one implementation, the histology-based digital staining system 102 creates a pixel-wise mask for the cell nuclei to provide an enhanced understanding of the composition and spatial organization in the pathology image 104. Stated differently, the histology-based digital staining system 102 generates a class label and bounding box coordinates for each nucleus in the pathology image 104 in addition to a mask. In one implementation, the histology-based digital staining system 102 extracts feature maps from the pathology images 104, which are passed through a Region Proposal Network (RPN). Application of the RPN predicts whether a nucleus is present in a region of the pathology image 104, such that candidate proposals are returned. The histology-based digital staining system 102 applies a region of interest (ROI) pooling layer and coverts all the proposals to the same shape, and the proposals are passed through a fully connected network to predict class labels and bounding boxes. Additionally, the histology-based digital staining system 102 generates a segmentation mask for each nucleus through a deconvolution layer. Combining the bounding boxes and the segmentation masks, the histology-based digital staining system 102 adds a mask for each region containing a nucleus. The masks for all the nuclei in the pathology image 104 are predicted to segment the nuclei of the cells, thereby providing segmented and classified nuclei, with which the characterized TME 106 and the prognostic model 108 are generated.

For the Mask R-CNN architecture of the histology-based digital staining system 102 to simultaneously segment and classify the cell nuclei in the pathology image 104, the histology-based digital staining system 102 is trained using training pathology images 110. In one implementation, the training pathology images 110 includes pathology images of different patient tissues manually labeled by expert pathologists. For example, pathology images 104 for lung adenocarcinoma (ADC) patients may be used to train the histology-based digital staining system 102 with the nuclei of tumor cells, stromal cells, lymphocytes, macrophages, red blood cells and karyorrhexis manually labeled by expert pathologists in the training pathology images 110.

Through training, the histology-based digital staining system 102 is automatically learned to identify different nuclei based on a wide range of feature maps, including color, size, and texture within neighborhood area of the pathology image 104. With the histology-based digital staining system 102 being trained, the pathology image 104 is simultaneously segmented and classified to identify cell types and cell spatial locations. From the identified cell types and cell spatial locations, cell spatial organization features may be derived by the histology-based digital staining system 102 to generate the characterized TME 106.

In some cases, TME-related image features may be significantly associated with patient overall survival. Thus, based on these image features, the characterized TME 106 is used to generate the prognostic model 108 for one or more patients. In one implementation, the prognostic model 108 includes a risk score 112 indicative of patient survival outcome. The risk score 112 may be associated with one or more risk groups into which a patient may be assigned. Using example lung ADC patients, the pathology model 108 was independently validated using pathology data, in which a predicted high-risk group showed significantly worse survival than a low-risk group (pv=0.001), with a hazard ratio of 2.23 [1.37-3.65] after adjusting for clinical variables. Furthermore, the image-derived TME features may be correlated with the gene expression of biological pathways based on the characterized TME 106. For example, transcription activation of both the T-cell receptor (TCR) and Programmed cell death protein 1 (PD1) pathways may be positively correlated with the density of detected lymphocytes in tumor tissues, while expression of the extracellular matrix organization pathway may be positively correlated with the density of stromal cells.

As discussed herein, the histology-based digital staining system 102 generates the characterized TME 106 based on the spatial organization of different types of cells in tumor tissues. Stated differently, the comprehensive nuclei segmentation and classification of the histology-based digital staining system 102 generates the characterized TME 106 from the pathology image 104, such as an H&E stained pathology image. The histology-based digital staining system 102 thus computationally stains different types of cell nuclei in the pathology image 104.

In one implementation, the histology-based digital staining system 102 segments the nuclei of tumor, stromal, lymphocyte, macrophage, karyorrhexis and red blood cells in lung ADC. In this example, the histology-based digital staining system 102 identified and classified cell nuclei and extracted 48 cell spatial organization related features that may be used to generate the characterized TME 106. Using the extracted features, the prognostic model 108 may be generated and the image-derived TME features correlated with the gene expression of biological pathways. As such, the histology-based digital staining system 102 dissect the TME from the pathology image 104 and uses the spatial organization of different cell types to predict patient survival and determine associations with the gene expression of biological pathways. In one example, the histology-based digital staining system 102 characterizes the tumor morphological microenvironment using tissue pathology images in lung ADC.

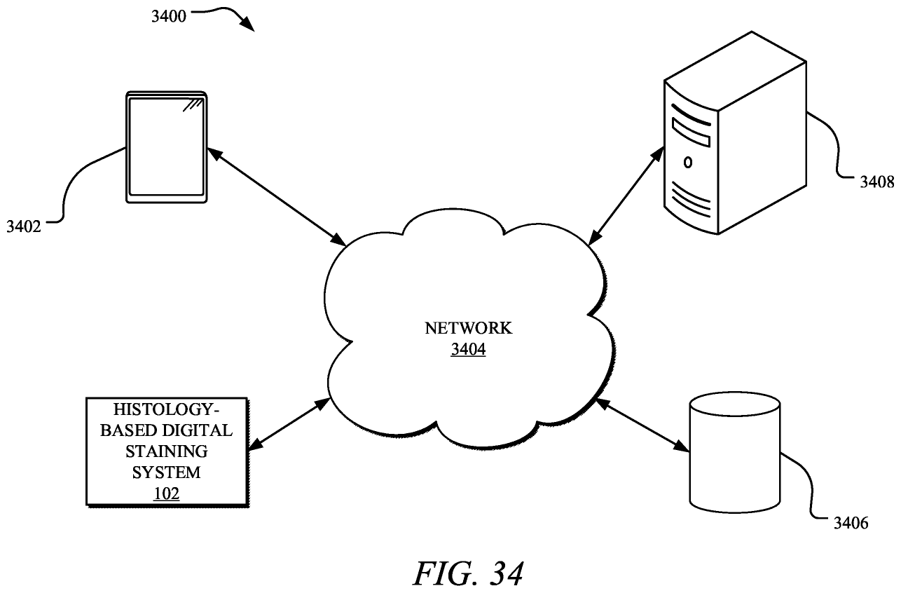

As described in more detail with respect to FIG. 34, in one implementation, the pathology image 104 is received by the histology-based digital staining system 102 over a network. Thus, in some cases, the histology-based digital staining system 102 may be accessible via a web-portal through which a user, such as a pathologist, may upload the pathology image 104 for analysis by the histology-based digital staining system 102. The histology-based digital staining system 102 may be used to analyze lung ADC pathology images, as wells as pathology images involving head and neck cancer, breast cancer and lung cancer squamous cell carcinoma pathology image datasets. The histology-based digital staining system 102 may be learned to handle different types of pathology images, cancers, and/or the like based on the training pathology images 110 and other training data.

In one implementation, the training pathology images 110 includes a training set 114, a validation set 116, and a testing set 118 of expertly labeled images. In one particular example, the training pathology images 110 includes 208 40× pathology images for 135 lung ADC patients were acquired from a first dataset, and 431 40× pathology images for 372 lung ADC patients from a second dataset, including multiple pathology images for a single patient. In this example, a specialized lung cancer pathologist manually labeled the tumor ROI for each of the training pathology images 110, with another lung cancer pathologist confirmed the labeling and another lung cancer pathologist annotating the lung ADC histology subtypes.

To construct the set of training pathology images 110 for the histology-based digital staining system 102 in this example, 127 image patches (500×500 pixels) from 39 pathological ROIs were extracted. In these patches, different types of cell nuclei were labeled. All the pixels within tumor nuclei, stromal nuclei, lymphocyte nuclei, macrophage nuclei, red blood cells, and karyorrhexis were labeled according to their categories and all the remaining pixels were considered “other.” These labels, also collectively called the mask for the Mask R-CNN, were then used as the ground truth to train the histology-based digital staining system 102. The labeled images were randomly divided into the training set 114, the validation set 116, and the testing set 118. To ensure independence among these sets 114-118, image patches from the same ROI were assigned together. More than 12,000 cell nuclei were included in the training set 114 (tumor nuclei 24.1%, stromal nuclei 23.9%, lymphocytes 29.5%, red blood cells 5.8%, macrophages 1.5%, karyorrhexis 15.2%), while 1227 and 1086 nuclei were included in the validation set 116 and the testing set 118, respectively in this example.

The Mask R-CNN of the histology-based digital staining system 102 is optimized for adapted pathological image analysis through customized data loader, image augmenter, image centering and scaling, and the histology-based digital staining system 102 that is pre-trained with a dataset and fine-tuned with the training pathology images 110. In one implementation, the training pathology images 110 are standardized (e.g., centered and scaled to have zero mean and unit variance) for each Red Green Blue (RGB) channel. Further, to increase generalizability and avoid bias from different H&E staining conditions, extensive augmentations on the image patches were performed for the training set 114. In particular, random projective transformations were applied to the training set 114 and the corresponding masks, and each image channel was randomly shifted using linear transformation. In one particular example, during the training process for the training set 114, a batch size was set to 2, the optimizer was to Stochastic Gradient Descent (SGD), the learning rate was set to 0.01 and decreased to 0.001 after 500 epochs, the momentum was set to 0.9, and the maximum number of epochs to train was set to 1000. In the validation set 116, the histology-based digital staining system 102 trained at the 707th epoch reached the lowest loss. This model was selected and used in the following analysis to avoid overfitting.

Since the histology-based digital staining system 102 simultaneously segments and classifies cell nuclei, three criteria were used to evaluate the segmentation performance in the validation set 116 and the testing set 118, respectively. First, detection coverage was calculated as the ratio between the detected nuclei and the total ground truth nuclei. Each ground truth nucleus was matched to a segmented nucleus, which generated a maximum Intersection over Union (IoU). If the IoU for a ground truth nucleus was >0.5, this nucleus was labeled “matched;” otherwise it was labeled “unmatched.” Second, nuclei classification accuracy was determined for the matched nucleus by comparing the predicted nucleus type with the ground truth. Third, segmentation accuracy was evaluated by the IoUs, which were calculated for each detected nucleus and averaged in different nuclei categories.

With the histology-based digital staining system 102 being trained, the histology-based digital staining system 102 may be used to generate the characterized TME 106 for the pathology image 104. In one implementation, the histology-based digital staining system 102 performs image feature extraction to describe nuclei composition and organization.

In some cases, to increase computational efficiency, among other reasons, such as retaining a good representation of each ROI, a pathology slide image is analyzed by the histology-based digital staining system 102 in patches, rather than through application to a whole slide. Thus, the pathology image 104 may correspond to a whole slide or a portion of the slide. In one particular example, 100 image patches (1024×1024 pixels) were randomly sampled and analyzed for each pathologist-labeled ROI. These 100 image patches provided good coverage of each ROI. Nuclei were then segmented and classified through the histology-based digital staining system 102.

To characterize the spatial organization of cells, the centroids of nuclei are calculated and used as vertices to construct a feature graph for each image patch of the pathology image 104. The feature graph provides a graphical representation of the nuclei within the pathology image 104 with each of the centroids of the nuclei represented as a vertex. The location of each vertex on the feature graph may be based on a spatial location of the nuclei within the pathology image 104.

In defining each feature graph, nearest neighbor information for each nucleus is generated. In one implementation, the nearest neighbor information is generated through Delaunay triangulation of the vertices within the feature graph of the pathology image 104. Delaunay triangulation generally involves a triangulation of a convex hull of points in a diagram in which every circumcircle of a triangle is an empty circle, such that for a given set P of discrete points in a plane is a triangulation DT(P) such that no point in P is inside the circumcircle of any triangle in DT(P). More particularly, for every three vertices in the feature graph, a circle is drawn through them. If the circle passes through three vertices and does not include any other vertices in the feature graph within the circle, the triangle formed by the three vertices is accepted as a valid triangle with edges of the triangle corresponding to connections between those vertices. Thus, for each vertex, the corresponding vertices connected with an edge within a triangle represent a nearest neighbor, such that there is no closer neighbor to which the vertex could have an edge. The Delaunay triangulation thus outputs a list of simplices, which detail the three vertices comprising each Delaunay triangle. In one implementation, in defining the edges between triangulated vertices, edges that connect each vertex are calculated by iterating through the simplices based on one or more edge attributes. A primary attribute of an edge may be a Euclidean or spatial distance between the two vertices it connects. It will be appreciated that the nearest neighbor connectivity of the nucleus may be obtained through other mechanisms in alternative or addition to Delaunay triangulation.

Stated differently, in one implementation, Delaunay triangulation is used to connect the nuclei into the feature graph, and the number of connections and the average length (i.e. spatial distance) between two types of nuclei summarize the spatial organization of different types of cell. As one example, the histology-based digital staining system 102 extracts features images according to six nucleus categories (tumor, stromal, lymphocyte, macrophage, karyorrhexis and red blood cell). In this example, the edges of the feature graph are classified into 21 categories [i.e. 6×(6+1)/2=21] according to their vertex pairs. For each pathology image 104 in this example, the number of connections (edges) for different categories are counted (21 features), the lengths of the connections are averaged for each edge category (another 21 image features), and the density of each type of nucleus are calculated (yielding 6 image features). In total, 48 image features are extracted in this example. The image features are averaged across the 100 patches for each ROI in the pathology image 104.

With the image features extracted from the pathology image 104 following simultaneous segmentation and classification of the cell nuclei, the histology-based digital staining system 102 generates the characterized TME 106, which may be used to generate the prognostic model 108. Overall survival, defined as the length of time from date of diagnosis till death or last contact, is used as the response variable for survival analyses by the histology-based digital staining system 102. In one implementation, the prognostic model 108 includes or is otherwise validated by a Cox Proportional Hazard (CoxPH) prognostic model for overall survival for lung ADC patients. Elastic-Net penalty may be used to avoid overfitting.

In the previous particular example, 22 features may be selected in the final CoxPH model. Given a set of the 22 image-derived TME features for each patient, the prognostic model 108 calculates the risk score 112 for the patient by summarizing the products between features and corresponding coefficients, with a higher risk score indicating worse prognosis. Based on the risk scores 112, patients may be dichotomized into predicted high-risk and low-risk groups using a median risk score as a cutoff.

In one implementation, the histology-based digital staining system 102 generates survival curves for each of the risk groups predicting prognosis over time. These survival curves may be estimated based on a Kaplan-Meier estimator survival analysis. However, other survival functions, such as proportional hazard models, and/or the like may be utilized. More particularly, the survival curves of the predicted high-risk and low-risk groups may be estimated using the Kaplan-Meier method. The survival differences between predicted high-risk and low-risk groups may be compared using a log-rank test. Additionally, in one implementation, a multivariate Cox proportional hazard model may be used to determine the prognostic value of predicted risk groups using image-derived TME features after adjusting for other clinical characteristics, including, without limitation, age, gender, smoking status, and stage.

As described herein, the histology-based digital staining system 102 may provide an indication of an association between the image features and gene expression of biological pathways. In one particular example, gene expression data of 372 patients were preprocessed: the genes whose mRNA expression levels were 0 in >20% of patient samples were removed. The correlation between mRNA expression levels and image-derived TME features may be evaluated using Spearman rank correlation, GSEA may be performed for each TME feature, and/or the like. For multiple testing correction, Benjamini-Hochberg (BH)-adjusted p values may be used to detect significantly enriched gene sets. Gene sets with BH-adjusted two-tailed p values<0.05 may be regarded as significantly enriched.

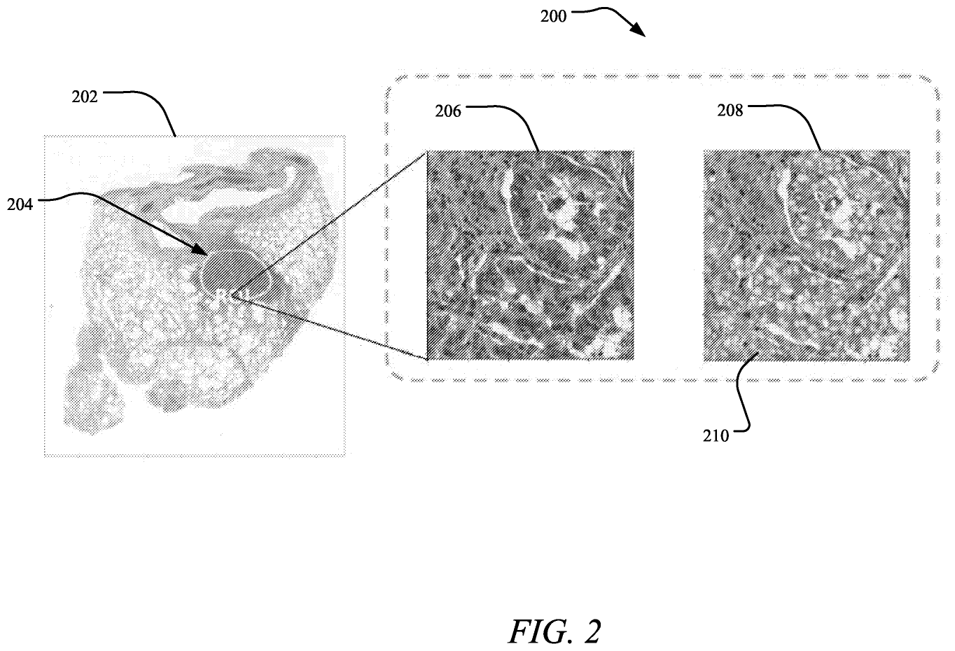

Turning to FIG. 2, an example output 200 of the histology-based digital staining system 102 is illustrated. More particularly, an example pathology image 202 includes an ROI 204. An image patch 206 is sampled from the ROI 204. Using the histology-based digital staining system 102, a nucleus segmentation 208 is generated from the image patch 206, including each nucleus 210 classified by cell type. From the nuclei segmentation 208, image feature extraction may be performed by the histology-based digital staining system 102 to generate the characterized TME 106 of the pathology image 202, as well as the prognostic risk score 112.

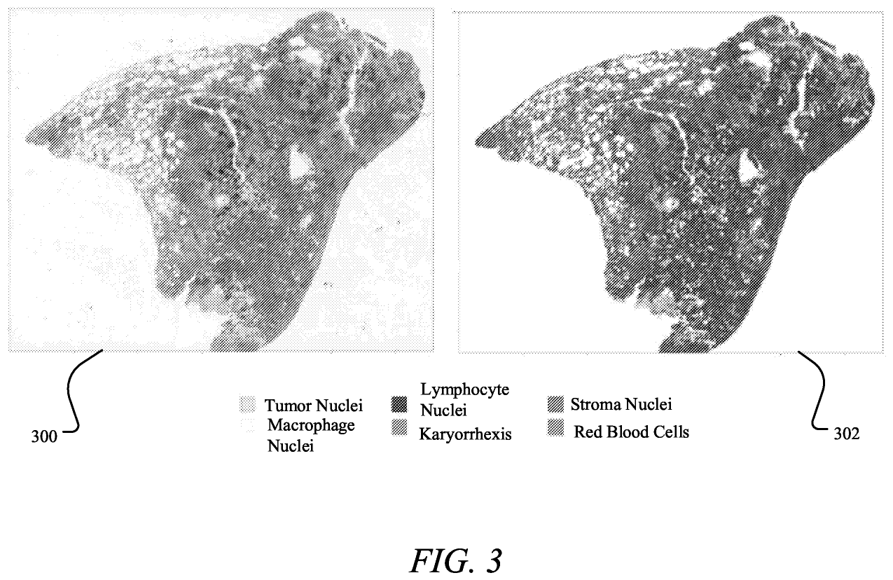

FIG. 3 shows an example of the pathology image 104 and the characterized TME 106 including a composition and a spatial organization of the patient tissue following cell nuclei segmentation and classification. Stated differently, the pathology image 104 includes a whole slide image 300 and generates a characterized image 302 following nuclei segmentation and classification with detected and classified nuclei overlaid on the whole slide image 300.

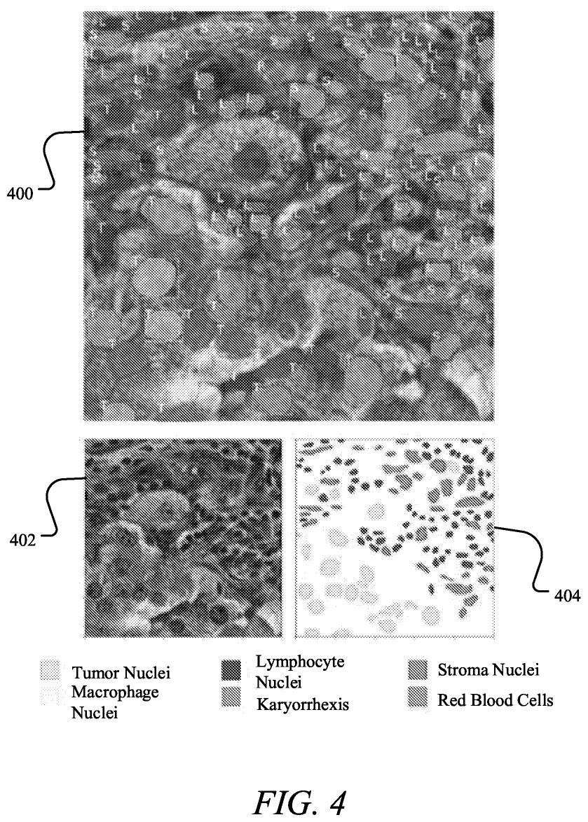

Turning to FIG. 4, segmentation and classification of cell nuclei is illustrated for an example of the pathology image 104 using the Mask R-CNN of the histology-based digital staining system 102. As shown in FIG. 4, a segmented image 400 is shown with an example pathology image 402 and extracted image features 404. With respect to the segmented image 400, each nucleus has a bounding box shown in dashed lines, with nucleus segmentation by the histology-based digital staining system 102 being performed within the bounding box. The class of nucleus is predicted by the histology-based digital staining system 102 at the same time as the segmentation, and the nucleus class is labeled proximate the bounding box.

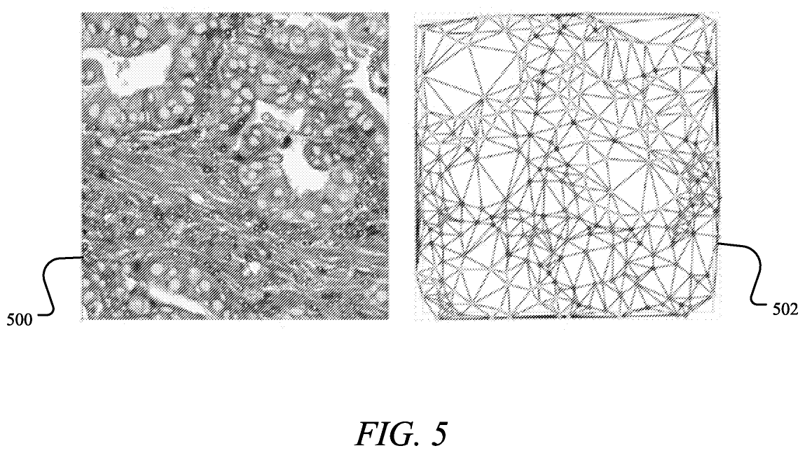

Referring to FIG. 5, an extraction of topological features from an example nuclei spatial organization to characterize the tumor microenvironment is illustrated. More particularly, nuclei segmentation results 500 from the simultaneous segmentation and classification of the nuclei by the histology-based digital staining system 102 includes centroids of the nuclei marked in white. A feature graph 502 is constructed through Delaunay triangulation using the nuclei centroids as vertices. To remove edge effect, when extracting graph properties, only edges with both ends within the solid gray square are counted as a primary attribute.

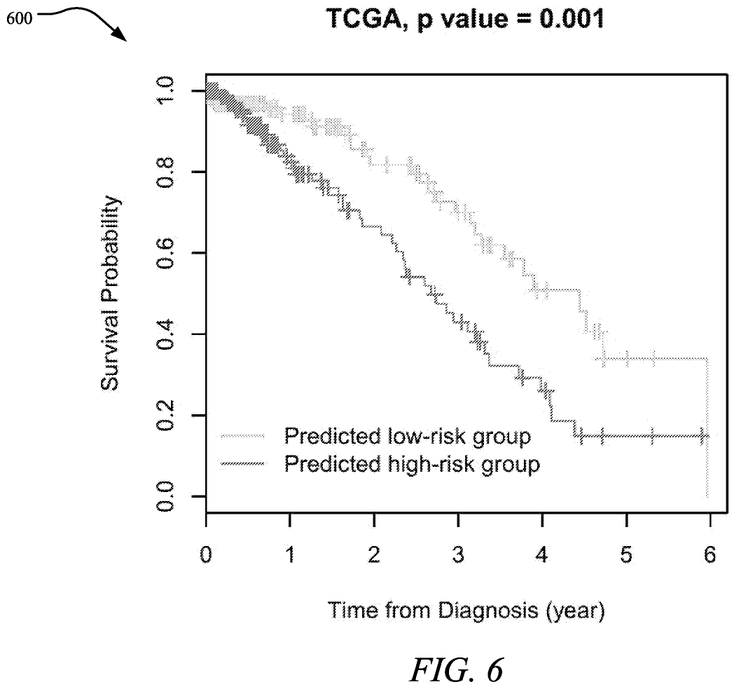

FIG. 6 shows an example plot 600 of predicted high-risk and low-risk groups generated based on the pathology model 108. In one implementation, the plot 600 provides a prognostic value of the TME-feature-based extraction of the prognostic model 108. The plot 600 may be survival curves estimated based on a Kaplan-Meier estimator survival analysis of high-risk and low-risk groups, with the log-rank test, p-value=0.001. As can be understood from the plot 600, patients in the high-risk group show significantly worse survival than those in the low-risk group.

In one particular example, TME features that were significantly correlated with survival outcome in univariate analysis show that higher karyorrhexis density, more karyorrhexis-karyorrhexis connections, and more karyorrhexis-red blood cell connections were associated with worse survival outcome, which is expected as these features indicate a higher rate of tumor necrosis. Furthermore, higher stromal nuclei density and more stromal-stromal connections are associated with better survival outcome, which is consistent with the observation that more stromal tissues correspond to better prognosis.

With respect to association between image features and transcriptional activity of biological pathways, GSEA was performed to identify the biological pathways whose mRNA expression profiles were significantly correlated with image-derived TME features. For example, the transcription activation of both the T-cell receptor (TCR) and Programmed cell death protein 1 (PD1) pathways may be positively correlated with lymphocyte density in the tumor tissue, which is consistent with reports that genes involved in the TCR and PD1 pathways are expressed in immune cells. In addition, expression of the extracellular matrix organization gene set, for which fibroblasts act as an important source, may be positively correlated with stromal cell density in tumor tissue.

Furthermore, GSEA shows that the cell cycle pathway was significantly enriched with genes whose expression levels were correlated with both the tumor nuclei density and karyorrhexis density in tumor issue. To look into the relationship between tumor cell density and the gene expression of the cell cycle pathway, the patients are grouped and sorted according to their tumor nuclei density. For each patient group, the average expression levels of genes within the cell cycle pathway and whose expression levels are significantly (p value<0.001) correlated with tumor nuclei density. Positive correlations between gene expression and tumor nuclei density can be observed for most of the cell cycle-related genes, except for one gene, POLD4, which showed an inverse trend. Most of the genes in the cell cycle pathway have higher expression in tumors with higher tumor nuclei density (may be a higher grade of tumor), while POLD4 shows the opposite pattern. This pattern of POLD4 compared with other genes in the cell cycle gene set is consistent with a previous study of lung cancer, while most cell cycle genes were upregulated in lung cancer, POLD4 is usually downregulated.

In one implementation, the system 100 includes the histology-based digital staining system 102 and the GCN system 120, which may be separate or integrated systems, providing a cell organization-based GCN utilizing spatial distribution and morphological features of detected nuclei, instead of an image patch, to classify cancer (e.g., lung cancer) histology subtypes. The system 100 constructs graph inputs of the cell organization-based GCN model through nuclei classification and segmentation using a neural network. The histology-based digital staining system 102 and/or the GCN system 120 are trained using the training pathology images 110, and partial derivatives with regard to input features may be used to interpret the cell organization-based GCN. The system 100 generally provides a hierarchical pathological analysis pipeline which feeds a computational staining result produced by the histology-based digital staining system 102 to the graph-based GCN model. The system 100 handles spatial distribution of nuclei in the TME. As described further herein, the cell organization-based GCN may be used in predicting response to EGFR targeted therapy. As the presently disclosed technology demonstrates, the cell organization-based GCN model of the GCN system 120 accurately distinguishes lung ADC versus SCC compared with traditional machine learning models. In validating the cell organization-based GCN model for EGFR targeted therapy response, EGFR TKI treated patients showed significantly improved survival outcome in a predicted benefitting group (p=0.0002) but not in a non-benefitting group (p=0.10).

In one implementation, the GCN system 120 handles nuclei distribution information in the pathology images 104. The system 100 uses features describing nuclei morphology and spatial arrangement in image recognition to provide a non-image based deep learning structure in the cell organization-based GCN of the GCN system 120. In general, the GCN system 120 uses a graph as an input, with a corresponding output being a predicted label of the graph, labels of graph vertices, features of a potential edge, and/or the like, depending on its specific structure. Since spatial distribution of nuclei on the pathology image naturally forms a graph, with nuclei acting as vertices and relative locations between a pair of nuclei in the Euclidean space acting as edges, using graphs to represent a noisy pathology image according to the presently disclosed technology reduces classification variance and improves accuracy. The histology-based digital staining and mask-RCNN of the histology-based digital staining system 102 providing nuclei classification and segmentation is used to automatically generate the spatial locations and morphological features for the cell organization-based GCN of the GCN system 120.

The GCN system 120 utilizes graph-represented images and GCN in cancer classification and treatment response prediction. For example, the GCN of the GCN system 120 may be applied to pathological nuclei networks to provide ADC classification and treatment response prediction. The system 100 constructs a cell graph utilizing the identification and classification of different cell types, as described herein. More particularly, the histology-based digital staining system 102 and the GCN system 120 are trained to identify and utilize, respectively, different cell types, such as tumor cells, stromal cells, lymphocytes, red blood cells, macrophages, karyorrhexis, and/or the like. In one implementation, the cell organization-based GCN is directly applied to image patches within tumor ROIs of the pathology images 104, and the system 100 generates information of each identified nucleus, including centroid position, cell type, confidence of prediction, nuclei orientation (defined as the angle between x-axis and major axis of nuclei), nuclei morphological features (area, convex area, eccentricity, extent, filled area, major axis length, minor axis length, perimeter square divided by area, perimeter, and solidity), and/or the like.

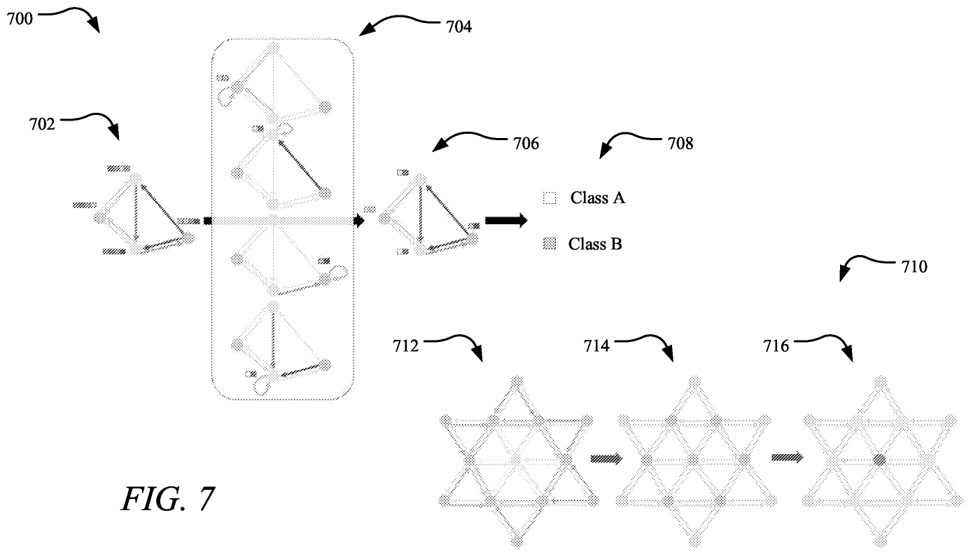

To continue a detailed description of graph construction, reference is made to FIG. 7. From the pathology image 104, the system 100 extracts one or more image patches for nuclei segmentation and generates computational staining using the histology-based digital staining system 102. The graph is constructed for graph classification using the GCN system 120. For example, the classification may include ADC and SCC. In one implementation, the system 100 provides graph construction using k-nearest neighbors. More particularly, the spatial distribution of all cells within an image patch permits a directed graph to be constructed for each image patch using k-Nearest Neighbors based on Euclidean distance, with the direction pointing to a cell from its neighbors.

As an example to illustrate these features, a diagram 700 of the EC convolution is shown in FIG. 7. An input graph 702 includes four nodes, with each node having a length-four feature vector. The input graph 702 is fed into an EC convolution layer 704. During EC convolution, each node receives and integrates messages from itself and its neighborhood. The message may be calculated from the node features as well as edge features of the input graph 702. An output 706 of the EC convolution layer 704 is an integrated message that becomes node features that act as inputs for a global pooling layer 706 from which classifications 708 are generated. The classifications 708 may include a first class (e.g., Class A) and a second class (e.g., Class B). Class A may be ADC, and Class B may be SCC, for example. A receptive field 710 of a central node may be generated. For example, a plurality of EC convolution layers may be used, with an input 712 generating a first output layer 714 after a first EC convolution layer. The first output layer 714 may be input to a second EC convolution layer to generate a second output layer 716. As can be understood from FIG. 7, if one EC Convolution is performed, the nodes within a one-hop connection contribute to the features in the first output layer 714. After a second EC convolution, the nodes within two-hop connections contribute to the features in the second output layer 716. The features of nodes and edges which do not contribute to the output features of the central node are colored in gray in FIG. 7.

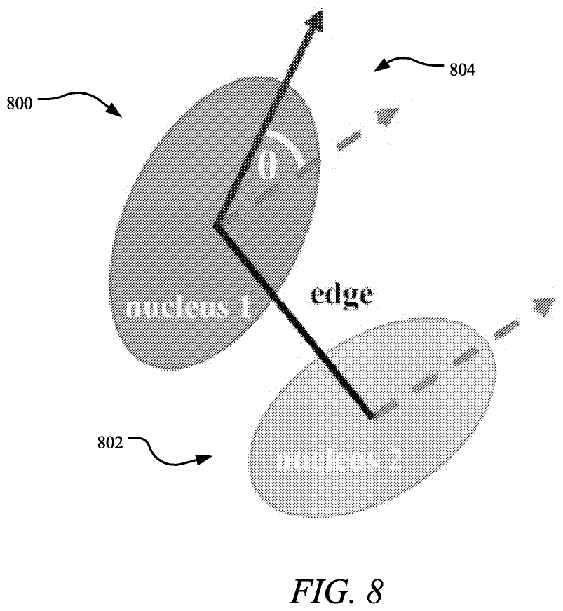

As such, in one non-limiting example, k is set to 8 to cover the adjacent neighbors of each nucleus. Each graph consists of two components: nodes (representing nuclei) and edges (representing spatial connections among nuclei). To further describe cell types and morphological features in graph, two feature matrices may be defined for nodes and edges, respectively. In one example, the node feature matrix contains eleven features: confidence of prediction and ten morphological features. The edge feature matrix contains three features: edge type based on cell types of starting node and ending node (yielding 6*6=36 edge types), edge weight defined as reciprocal of Euclidean edge length, and edge angle. As shown in FIG. 8, the edge angle may be defined as cosine of an angle between major axes of the starting node and ending node. For example, a cosine of an angle 804 corresponding to an orientation between a first nucleus 800 and a second nucleus 802 may provide the edge angle. The features are globally centered and scaled before feeding into the GCN system 120.

The graph representation of nuclei spatial distribution and orientation distinguishes among histological subtypes of cancer, such as the two main histological subtypes of lung cancer, ADC and SCC, without directly using high-dimensional images, thereby decreasing computational burden while increasing accuracy. In one non-limiting example, the cell organization-based GCN of the GCN system 120 is constructed with three EC convolution layers followed by a subgroup mean pooling layer and a softmax layer. A disconnected graph is input as an input layer to the three EC convolution layers. The output from the EC convolution layers is input into a global mean-pooling layer. Through the global mean pooling layer, only features for tumor nuclei nodes are averaged. The output from the global mean pooling layer is input into the softmax layer, which outputs an output layer providing a probabilities of belonging to ADC and SCC.

The classification cell organization-based GCN model is trained, validated, and tested using the training pathology images 110. In one implementation, in constructing the training pathology images 110, one or more image patched are extracted from ROI of each pathology slide and transformed into graphs. In one example, graphs that were not sufficiently informative to be classified as ADC or SCC may be discarded from the training pathology images 110. The remaining graphs may be assigned to one or more of the training set 114, the validation set 116, and the testing set 118. To avoid data leakage, graphs from the same pathology slide may be assigned to the same set 114-118.

In one non-limiting example, the training pathology images 110 include 100 image patches with a resolution of 1024*1024 under 40× magnification. The image patches are extracted from ROI of each pathology slide and transformed into 100 graphs individually. Only graphs containing at least 20 tumor cells may be considered as informative enough to be classified as ADC/SCC and included in the training pathology images 110. In this example, the training pathology images 110 contain 40,971 graphs from a first source and 33,998 from a second source. The graphs from each of the sources are assigned to one or more of the training set 114, the validation set 116, and the testing set 118. For example, from the first source, 28,522 graphs from 328 slides may be assigned to the training set 114, 3,985 graphs from 46 slides may be assigned to the validation set 116, and 8,464 graphs from 95 slides may be assigned to the testing set 118. From the second source, 23,667 graphs from 265 slides may be assigned to the training set 114, 3,353 graphs from 37 slides may be assigned to the validation set 116, and 6,978 graphs from 77 slides may be assigned to the testing set 118. Graphs from the same slide are assigned to the same dataset to avoid data leakage. A third source containing 24,204 graphs from ADC patients and 12,196 graphs from SCC patients may be assigned to the testing set 118.

To train the GCN system 120 for ADC versus SCC classification, cross-entropy may be used as loss function. Additionally, a Stochastic Gradient Descent may be used as optimizer, for example with learning rate=0.0001 and momentum=0.9. In one example, the maximum training epoch is set as 300, and the model at the 285^{th} epoch with highest classification accuracy in the validation dataset is selected and applied to datasets of the training pathology images 110. Loss decreases and classification accuracy increases with the training process. Major voting among all graphs from labeled ROI is used to determine the histology subtype of a slide.

In one implementation, the cell organization-based GCN of the GCN system 120 has an input layer ID i, node set N, node features X(i) ϵR^{n×(i)}, edge set E_{n,m} (m ϵ neighbors of n), edge features E ϵR^{e×3} (edge type t_{n,m}, edge weight w_{n,m}, edge angle a_{n,m}), and channel number of output layer c. For each n in the node set N, the cell organization-based GCN of the GCN system 120 calculates a message from neighbors, calculates a message from a root node, aggregates the messages from the input layer, and performs activation and training-specific dropout layer using a rectified linear unit (ReLU). In calculating the message from neighbors for each n, for m in neighbors of n, the cell organization-based GCN of the GCN system 120: embeds t_{n,m} to edge modulator mo_{n,m}ϵ R^{(x×c)} through edge type embedding EM ϵR^{36×(x×c)}; updates mo_{n,m} with w_{n,m}, a_{n,m} through feature-wise linear modulation; reshapes mo_{n,m n,m}R^{(x×c)} as mo_{n,m} ϵR^{x×c}; and calculates message me_{n,m}=Xn×mo_{n,m}; me_{n,m} ϵR^{1×c}. The cell organization-based GCN of the GCN system 120 calculates the message from the root node with meₙ=Xₙ×θⁱ; meₙ ϵ R^{1×c}. The cell organization-based GCN of the GCN system 120 aggregates the messages from the input layer i with output features: X(i+1)ₙ=meₙ+Ave(me_{n,m}). The cell organization-based GCN of the GCN system 120 generates activation and training-specific dropout layer: X(i+1)ₙ=Dropout(ReLU(X(i+1)ₙ), training flag).

In one implementation, the system 100 predicts treatment response to EGFR TKI targeted therapy using a deep learning pathology image analysis pipeline. The cell organization-based GCN of the GCN system 120 generates a prognosis for a patient by providing all connected graphs from the pathology image 104 in a single disconnected graph. The disconnected graph is input at the input layer into a plurality of EC convolutional layers and output into a global mean-pooling layer. The global mean pooling layer evaluates all cell types within the TME. The output from the global mean pooling layer is input into the softmax layer, which outputs an output layer providing a probability of high-risk for the prognostic model 108. Nuclei morphological features may be set to 1 to be excluded from the input graph in generating a response prediction using the GCN system 120.

The cell organization-based GCN of the GCN system 120 may be trained using the training set 114 and validated using the validation set 116. In one example, the training set 114 and the validation set 116 each include biopsy images and clinical information for patients carrying the EGFR mutation. In one particular example, the training set 114 contains 115 biopsy slides from 98 patients, and the validation set 116 contains 139 biopsy slides from 136 patients. In this example, patients with short (OS≤31 month, #slides=55) or long (OS>31 months, #slides=49) survival outcome are assigned to non-benefitting and benefitting group, respectively. The cutoff is selected to ensure non-benefitting and benefitting groups are balanced. Overall survival (OS), defined as time from diagnosis of metastatic disease to death or last follow-up, is used as outcome. Table 1 shows patient characteristics for the training set 114 and the validation set 116.

TABLE 1

| | Training | | Non- | | | | P |
|---|---|---|---|---|---|---|---|
| | Benefitting | | benefitting | | Validation | | value |
| # EGFR mutated | 50 | | 48 | | 136 | | |
| # EGFR Ttx | 50 | | 48 | | 98 | | |
| treated | | | | | | | |
| # Biopsy slides | 64 | | 51 | | 139 | | |
| Age (year) | 62.4 ± 9.6 | | 59.5 ± 10.4 | | 62.5 ± 10.3 | | 0.20 |
| Gender (%) | | | | | | | 0.029 |
| Male | 8 | (16%) | 11 | (23%) | 47 | (35%) | |
| Female | 42 | (84%) | 37 | (77%) | 89 | (65%) | |
| Smoking | | | | | | | 0.11 |
| status (%) | | | | | | | |
| Current | 0 | (0%) | 3 | (6%) | 4 | (3%) | |
| Former | 23 | (46%) | 13 | (27%) | 60 | (45%) | |
| Never | 27 | (54%) | 32 | (67%) | 70 | (52%) | |
| Surgery | | | | | | | 0.0011 |
| received? (%) | | | | | | | |
| No | 18 | (36%) | 35 | (73%) | 90 | (66%) | |
| Yes | 32 | (64%) | 13 | (27%) | 42 | (31%) | |
| Unknown | 0 | (0%) | 0 | (0%) | 4 | (3%) | |
| Stage at | | | | | | | 0.046 |
| diagnosis (%) | | | | | | | |
| I | 6 | (12%) | 2 | (4%) | 5 | (4%) | |
| II | 3 | (6%) | 2 | (4%) | 3 | (2%) | |
| III | 11 | (22%) | 7 | (15%) | 11 | (8%) | |
| IV | 29 | (58%) | 36 | (75%) | 112 | (82%) | |
| Unknown | 1 | (2%) | 1 | (2%) | 5 | (4%) | |

To train response prediction of the GCN system 120 in this example, cross-entropy may be used as a loss function and an adaptive deep learning rate with scaling factor of 2 may be used as the optimizer. A maximum training epoch is set as 300, and the model at the 135^{th} epoch with a highest classification accuracy in the training set 114 is selected. The probability of belonging to the benefitting group is used as a benefitting score. In the testing set 114, patients are dichotomized into the benefitting and non-benefitting groups according to the median benefitting score. Kaplan-Meier curves and log-rank tests may illustrate the survival difference between EGFR TKI treated and non-treated patients in the benefitting group and non-benefitting group, respectively. The differences are considered significant when two-tailed p-value<0.05.

Overall, the fit of the geometric distribution of nuclei into a graph landscape optimizes pathology image recognition, outperforming traditional image-based deep learning models. While classification of ADC versus SCC is described herein as an example, it will be appreciated that the GCN-based prediction may be applied to various types of classification. As described herein, the system 100 provides an automatic pathology image analysis pipeline consists of image patch extraction from tumor ROI, classification and segmentation of nuclei with HD-Staining, graph construction, and GCN-based prediction. The cell organization-based GCN of the GCN system 120 produces one label for each input graph, and the labels are summarized using major voting to determine slide-level label.

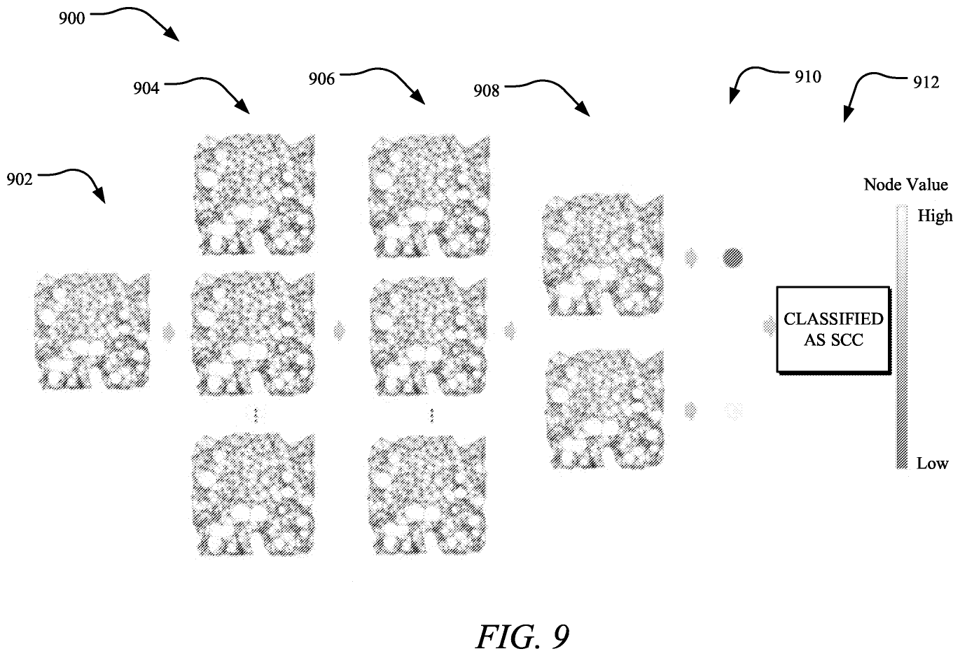

In one implementation, the system 100 provides histology classification with image patch level labels. Through weakly supervised learning, the image patch level labels are provided to the GCN system 120 to predict cellular level classes. As such, in one example, the system 100 visualizes a cellular level predicted score for both ADC and SCC, thereby providing an understanding of how different nuclei spatial distribution patterns affect the histological determination by the cell organization-based GCN of the GCN system 120. Referring to FIG. 9, in one implementation, an example GCN structure 900 includes an input graph 902, which is passed through EC convolution layers 904-908, each with n channels. A first EC convolution layer 904 and a second EC convolution layer 906 may each have ten channels and a third EC convolution layer 908 may have two channels. By calculating such a length—2 feature vector after the 3third EC convolution layer 908 and a subgroup mean pooling layer 910 all cell types within the TME are evaluated and a classification 912 is output.

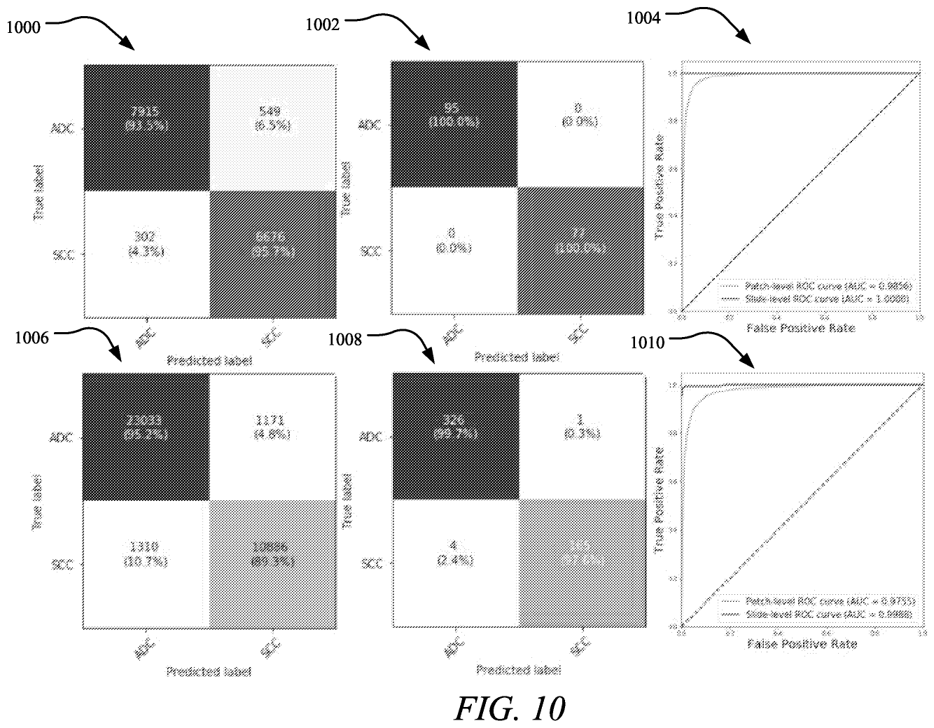

FIG. 10 illustrates example performance of the GCN system 120 in histology subtype classification. FIG. 10 shows an evaluation of a performance of the cell organization-based GCN in both image patch level and slide level. An image patch level confusion matrix 1000, a slide level confusion matrix 1002, and a receiver operating characteristic curve (ROC) curve 1004 for lung histology type classification are shown for a first testing set. Additionally, a second image patch level confusion matrix 1006, a second slide level confusion matrix 1008, and a second ROC curve 1010 for lung histology type classification are shown for a second testing set. In the first testing set, the patch-level accuracy is 94.5% with 0.986 Area under Curve (AUC). The slide-level accuracy is 100% with 1.000 AUC. In the second testing set, the patch-level accuracy is 93.2% with 0.976 AUC. The slide-level accuracy is 99.0% with 0.999 AUC.

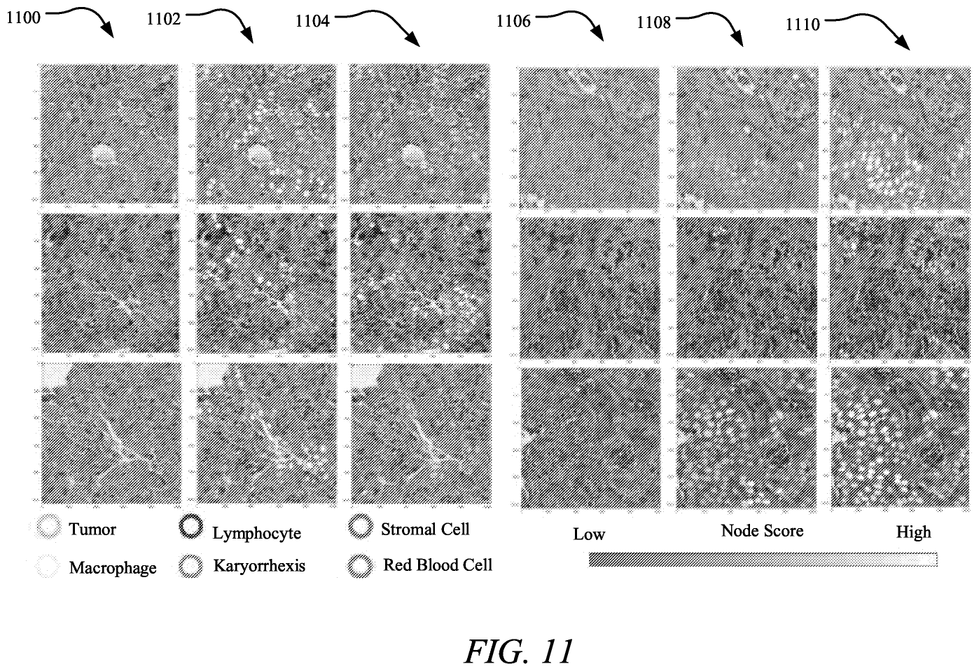

In performing lung cancer pathology subtype classification, the nuclei graph-based GCN model of the GCN system 120 improves classification performance through increased image recognition accuracy. The cellular level classification of ADC vs. SCC histology subtype is shown in FIG. 11. HD staining images 1100 and 1006, ADC score images 1102 and 1108, and SCC score images 1104 and 1110 are shown. As shown in FIG. 11, the GCN system 120 provides visualizing contribution of each tumor nuclei to final histological classification. To better illustrate nuclei features, mask of each cell is reconstructed from nuclei morphological features extracted by HD staining, with yellow representing a higher score and green a lower score. The GCN system 120 averages the tumor cell ADC scores and SCC scores, respectively, such that the uneven neuron activation heatmap highlights the typical patterns of nuclei distribution for ADC and SCC.

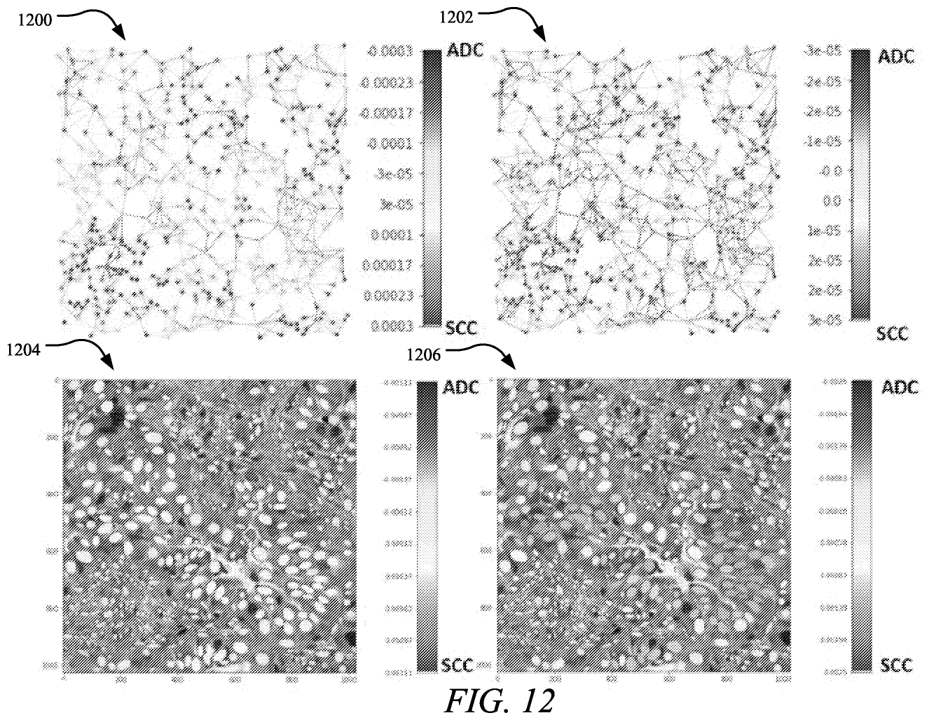

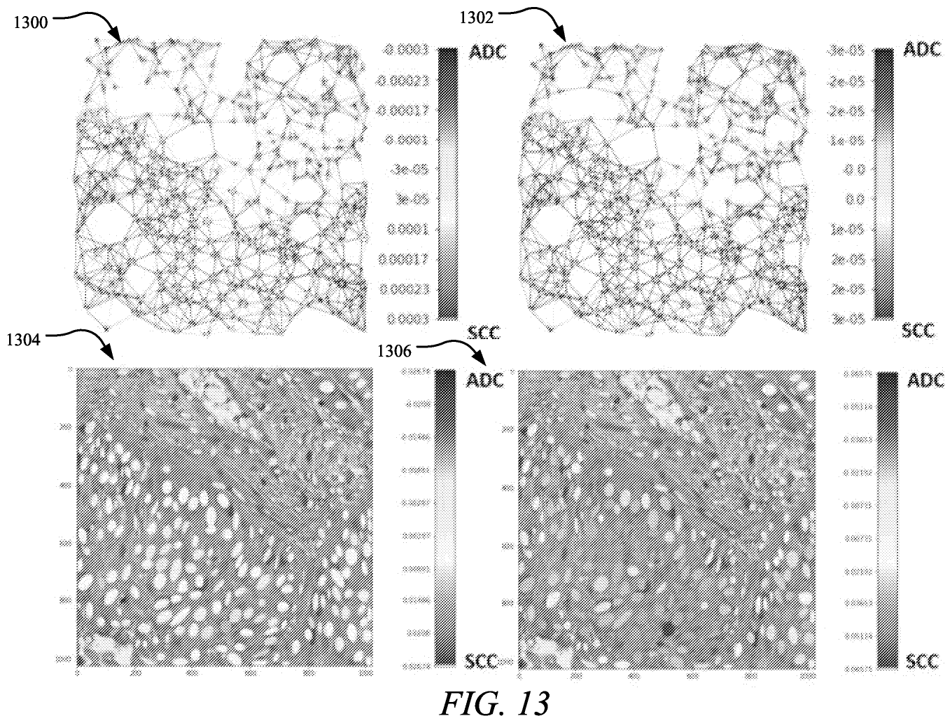

Turning to FIGS. 12-13, contribution of individual input features to final ADC vs. SCC classification of a nuclei graph is visualized, demonstrating an effect of nuclei distribution patterns on cell organization-based GCN prediction. FIG. 12 illustrates an edge angle plot 1200, an edge weight plot 1202, an eccentricity plot 1204, and a solidity plot 1206 for an example ADC image patch. Similarly, FIG. 13 illustrates an edge angle plot 1300, an edge weight plot 1302, an eccentricity plot 1304, and a solidity plot 1306 for an example SCC image patch. In the examples of FIGS. 12 and 13, gradients with respect to edge attributes are colored in the graph edges for optimized visualization. Additionally, centroid of nuclei are colored with respect to cell types generated by staining using the histology-based digital staining system 102. In these example plots, bluer color represents an increasingly negative gradient, indicating a trend towards ADC subtype.

In FIGS. 12-13, partial derivatives of predicted ADC (defined as cross-entropy between cell organization-based GCN output and a presumptive ADC label) with regard to the input features are used to represent the effect of input disturbance to classification output. Tumor cells tend to have higher eccentricity and solidity in the SCC subtype than in the ADC subtype. Consistent with this, the examples of FIGS. 12-13 illustrate that higher eccentricity and solidity of tumor cells in both the ADC plots 1200-1206 and the SCC plots 1300-1306 attribute to classification as SCC, while the contributions of eccentricity or solidity of other cell types are generally smaller than tumor cells. Additionally, as can be understood from FIG. 12-13, both of the edge attributes, edge angle and edge weight, contribute to the pathology subtype prediction. As previously discussed, edge angle corresponds to a similarity of nuclei directions and edge weight corresponds to an inverse of distance between nuclei centroids. Such a visualization is coherent with pathological observations that tumor cells in SCC subtype have a more structured architecture than in ADC subtype.

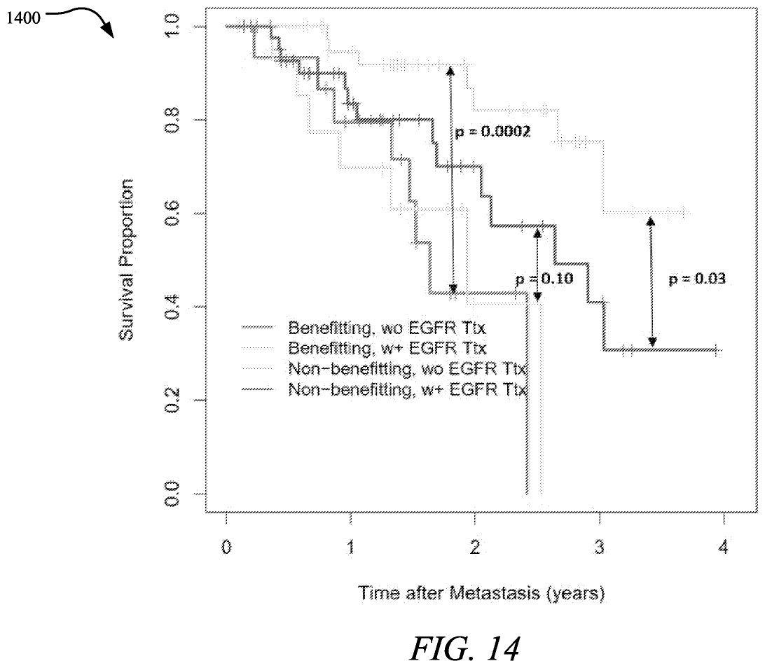

The system 100 provides GCN-based pathology image analysis for histological classification in a variety of contexts. As described herein, in one example, the system 100 predicts responsiveness to EGFR TKI targeted therapy. Turning to FIG. 14, in one example, to predict a slide-level benefitting score, all image patches from the same pathology slide are grouped together to construct one disconnected graph. The GCN system 120 is trained to predict a benefitting score for each input graph and applied to patients with EGFR mutation in the testing set 118. As shown in a plot 1400 of survival proportion and time after metastasis, within the predicted benefitting group, patients who did not receive EGFR TKI targeted therapy showed significantly worse survival than patients who received EGFR TKI targeted therapy. As shown in the plot 1400, p=0.0002; without targeted therapy versus with targeted therapy, with a Hazard Ratio [HR]=6.81, and a 95% Confidence Interval [CI] 2.14-21.73. In contrast, within the predicted non-benefitting group, there was no significant survival difference between targeted therapy treated and non-treated patient groups. As shown in the plot 1400, p=0.10; without targeted therapy versus with targeted therapy, HR=2.22, and 95% CI 0.85-5.83. As further illustrated in Table 2, after adjusting for potential clinical confounders, including age, gender, smoking status, surgery, and stage at diagnosis, a high benefitting score calculated by the GCN system 120 is predictive for prolonged overall survival in patients who carried the EGFR mutation and received EGFR TKI targeted therapy.

TABLE 2

| | | | p |
|---|---|---|---|
| Feature | HR (95% CI) | | value |
| Benefitting group stratified with Ttx | | | |
| Benefitting, w+ Ttx | 1 | (reference) | — |
| Benefitting, wo Ttx | 4.60 | (1.44-14.66) | 0.010 |
| Non-benefitting, w+ Ttx | 2.80 | (1.08-7.28) | 0.034 |
| Non-benefitting, wo Ttx | 6.44 | (1.91-21.73) | 0.003 |
| Age (per year) | 0.99 | (0.96-1.03) | 0.75 |
| Smoking status | | | |
| Current smoker | 1 | (reference) | — |
| Former smoker | 0.32 | (0.06-1.69) | 0.18 |
| Never smoker | 0.18 | (0.03-0.98) | 0.047 |
| Gender (male vs. female) | 0.81 | (0.37-1.77) | 0.60 |
| Surgery (w+ vs. wo) | 0.61 | (0.24-1.53) | 0.29 |
| Stage at diagnosis | | | |
| Stage I | 9.3 | (1.64-49.58) | 0.011 |
| Stage II | 2.41*10⁻⁷ | (0.00-Infinite) | 1.00 |
| Stage III | 1.00 | (0.29-3.53) | 1.00 |
| Stage IV | 1 | (reference) | — |

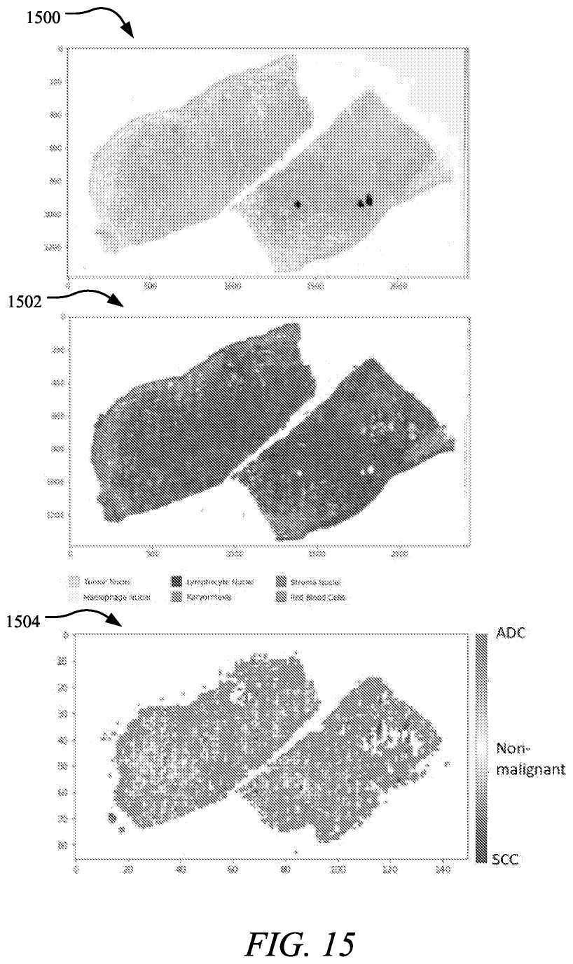

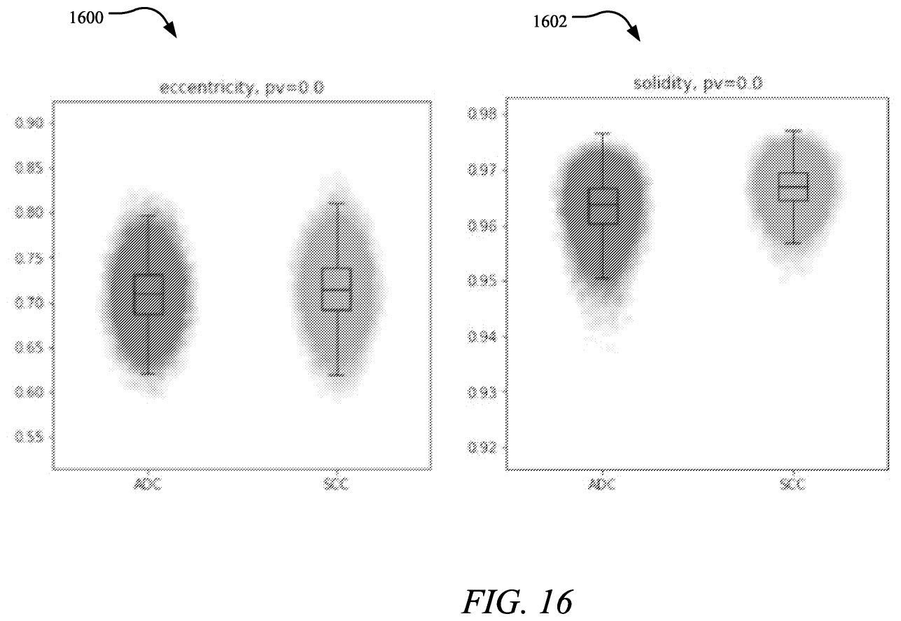

Turning to FIG. 15, it will be appreciated that the system 100 provides high accuracy pathology subtype classification for a whole pathology slide 1500. By sliding the window of the histology-based digital staining system 102 and the GCN system 120 across the whole pathology slide 1500, a characterized image 1502 with detected and classified nuclei overlaid on the whole pathology slide 1500 is generated. The GCN system 120 utilizes the characterized image 1502 in generating a whole-slide GCN prediction 1504, predicting pathology subtype classification. For example, as shown in FIG. 15, the whole-slide GCN prediction 1504 labels ADC with red, SCC with blue, and non-malignant with white. In generating the pathology subtype classification, the GCN 120 leverages the tendency of tumor cells to have higher eccentricity and solidity in SCC subtype than in ADC subtype, as illustrated in an eccentricity plot 1600 and a solidarity plot 1602 of FIG. 16.

The system 100 provides a hierarchical pathological analysis pipeline, which feeds a computational staining result produced by the histology-based digital staining system 102 to the GCN system 120 to provide histology classification and predict treatment responsiveness through characterization of a spatial distribution of nuclei in the TME. For example, the GCN 120 may be used to predict responsiveness to EGFR treatment.

The system 100 optimizes classification, with significantly reduced parameters compared with other systems. For example, the system 100 contains under 10,000 parameters, which is approximately 1/5000 of other models. The reduced amount of parameters increases portability, computational load, and interpretability.

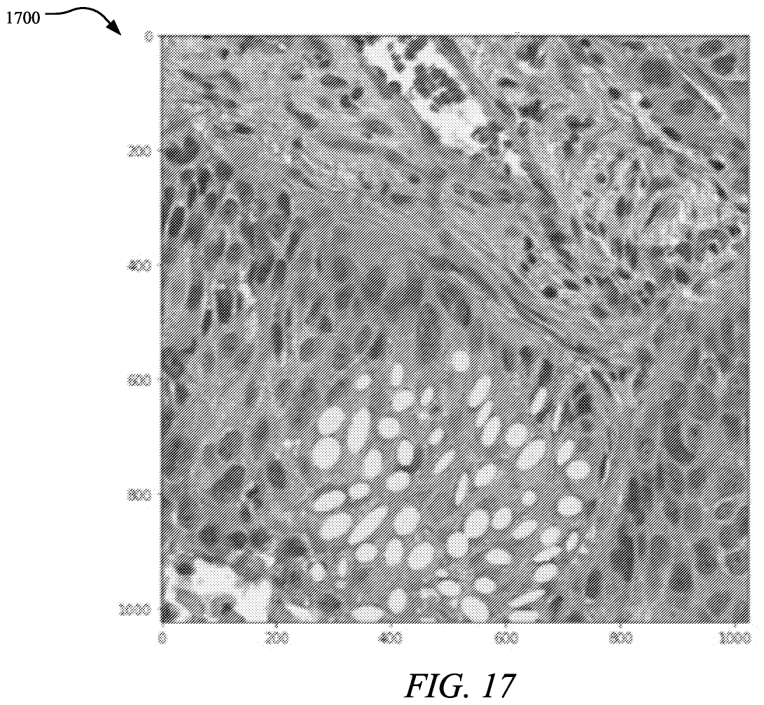

The receptive field for ADC vs. SCC classification is relatively small and flexible. In one example, instead of utilizing a 512×512 pixels image under 5× magnification, which represents a square area of 4096×4096 pixels image under 40× magnification, the GCN system 120 involves a small area under 40×. According to a receptive field of a single node 1700 as shown in FIG. 17, nuclei graph from any irregular area can be used as an input to the GCN system 120 as long as it covers enough spatially connected nuclei.

As demonstrated herein, using the cellular distribution alone, the system 100 provides cancer histology subtype recognition. The GCN system 120 uses the nuclei morphological and distribution features as input based on pathological knowledge. Additionally, the system 100 may characterize cytosolic or plasma membrane features in addition or alternative to utilizes the cellular architecture for subtyping. Cytosolic may be used, for example, to distinguish signet ring ADC and mucinous ADC, and detection of obvious plasma membrane is also a marker for SCC. Although the morphological features and distributions of nuclei are informative to distinguish ADC from SCC and to predict treatment response to EGFR TKI targeted therapy, as demonstrated herein, combining cytosolic and membranous features may provide additional insight.

The system 100 provides classification and segmentation for advanced pathological image analysis, including tumor classification, and nuclei segmentation. As described herein, the classification performed by the system 100 classifies given images, patches, or slides into predefined categories, and the segmentation performed by the system 100 identifies segments (e.g., pixels) of interests. In some instances, the segmentation may be categorized into two types: semantic segmentation assigning labels to every pixel of the given image; and instance segmentation further separating multiple objects of the same class as distinct objects.

Although some deep-learning algorithms perform well in many preset computational challenges in the biomedical space, the performances of such algorithms often significantly deteriorate when applied to digital pathology images, due to the diversity of pathological images. For example, the differences between scanner type, manufacturer and digitization process can restrain the quality of digital pathology slides in terms of image sharpness, resolution, noise level, amplification magnitude, and/or the like. In the context of H&E-stained histopathology images analysis described herein, the color variation provides a unique challenge, which can be impacted by stain concentration, time elapsed, environmental temperatures upon staining, and/or other factors. Without properly accounting for these image quality issues and staining variations, classification, segmentation, and characterization may have decreased accuracy. In other words, inadequate image quality, low amplification magnitude, and staining variation may decrease an accuracy of tumor region segmentation, nuclei detection, and classification. Thus, the system 100 may perform image restoration and quality enhancement using the image restoration system 122 to restore blurred regions, enhance low resolution/magnification into high resolution, normalize staining colors to reduce staining variation, and/or the like.

Blurred patches/regions reduce the image quality and thus decrease the performance of classification and semantic segmentation that involve local structure and details. Nuclei segmentation for lung cancer, for example, is very sensitive to image quality and resolution to correctly classify and segment small nuclei using fine details of an image. Moreover, small crowded objects may limit performance of instance segmentation algorithms utilizing Mask R-CNN, limiting an ability to transfer learning a low magnitude model (e.g., 10×, 20×) from a robust high magnitude model (e.g., 40×). Similarly, by not accounting for staining variance, alternative color normalization can potentially reduce the risk of low performance. Thus, the image restoration system 122 compensates for the weakness of existing deep learning models and can release the burden of transfer learning by improving quality and normalizing the staining variation.

The image restoration system 122 provides high quality in both realistic and resolution. In one implementation, the image restoration system 122 utilizes Generative Adversarial Network (GAN) including a first subnetwork and a second subnetwork. The first subnetwork may be a generator network and the second subnetwork may be a discriminator network. During training, the generator network learns to generate fake images that are hard to distinguish from real images by the discriminator network. Meanwhile the discriminator network distinguishes the generated fake images from real images. Once competition between the discriminator network and the generator network reaches equilibrium, the generator network is configured to produce like-real images that are virtually impossible for the discriminator network to identify. Unlike conventional generative models that sample from an explicitly learned distribution from data, the image restoration system 122 skips the process of fitting distributions, while implicitly mastering direct sampling. The image restoration system 122 thus generates GAN synthesized images, providing fine local structures and details with multiple enhancing tasks in one model without prior assumptions.

More particularly, the image restoration system 122 provides a deep-learning based image enhancer and restoration tool to enhance the quality of pathological images. The image restoration system 122 restores blurred regions, enhances image resolution, and reduces staining variation. The output of the system 100 in characterization the TME and predicting prognosis improves in training the system 100 with high quality images generated by the image restoration system 122. Furthermore, the image restoration system 122 is a lightweight model that can enhance a batch of images almost instantly (e.g., an average 0.145 s to 2.0 s per batch). The image restoration system 122 is a powerful tool to enhance image quality and improve the stability of the systems 102 and 120, as well as other deep learning algorithms in the system 100.

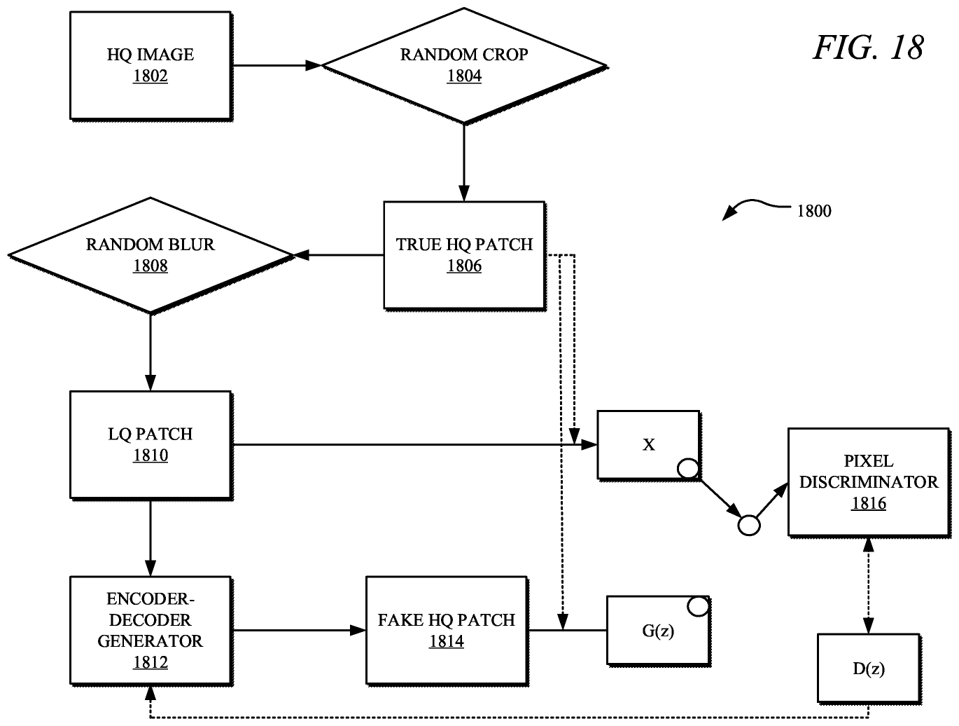

Turning to FIG. 18, an example image restoration architecture 1800 of the image restoration system 122 is shown. In one implementation, a high-quality (HQ) image 1802 are input through a random crop 1804 to form a batch of small true high-quality (HQ) patches 1806.

The batch of true HQ patches 1806 is input through a random blur 1808 to generate locally or globally randomly blurred low quality (LQ) image patches 1810. An encoder-decoder generator based on a residual neural network 1812 generates a batch of fake HQ image patches 1814 using the LQ image patches 1810. The LQ image patches 1810 and the fake HQ image patches 1814 are fed into a pixel-level least-square GAN (LSGAN) discriminator 1816 conditional to the original ground truth patch of the true HQ patch 1806. The formula utilized to update the discriminator D may be:

$${min}_{D} {V}_{LSGAN} ( D ) = \frac{1}{2} {𝔼}_{x \sim Pdata ( x )} [ {( D ( x ) - 1 )}^{2} ] + \frac{1}{2} {𝔼}_{z \sim {p}_{z} ( z )} [ {( D ( G ( z ) ) )}^{2} ] \vert$$

The formula utilized to update the generator G may be:

$${min}_{G} {V}_{LSGAN} ( G ) = \frac{1}{2} {𝔼}_{z \sim {p}_{z} ( z )} [ {( D ( G ( z ) ) - 1 )}^{2} ]$$

The image restoration architecture 1800 may utilize a pixel to pixel framework. For a generator of the encoder-decoder generator 1812, a residual network backbone may be used as the encoder structure and with a transpose convolution layer with pixel shuffle in a decoder of the encoder-decoder generator 1812. The pixel shuffle layers can help avoid checkerboard artifacts in generated images. For the pixel discriminator 1816, a 3-layers-pixelGAN may be used, having fewer parameters, thereby expediting the training process and providing sharper borders and enhance resolution.

With respect to loss functions, when updating the pixel discriminator 1816, a combination with the following losses may be used: (1) mse adversarial loss, (2) pixel-space mse loss [49], (3) content-space Euclidean distance from a feature map, (4) and total variation (TV) loss with the given weights: 0.001:1:0.006:2e-8:

L_{G}=0.001×*L_{adv}+1.0×Lₚᵢₓₑₗ+0.006×L_{perceptual}+2e⁻⁸×L_{TV}

In one implementation, during training, several HG images go through the random crop 1804 to generate a batch of the true HG patches 1806. The HQ patches 1806 are paired with the LQ image patches 1810 that are randomly distorted using the random blur 1808. The random blur 1808 may use one or more of the following distortions to simulate different conditions: (1) to simulate the effect of blurring, the random blur 1808 globally/locally blurs the image with Gaussian blur, median blur, and/or motion blur; (2) to simulate the effect of reduced-resolution, the random blur 1808 shrinks the image with a random scale (e.g., 0.25×-0.5×) under a different interpolation level (nearest, bi-linear, bi-quadratic, bi-cubic, etc.) and resizes it back to original size with bi-cubic interpolation; and (3) to simulate the staining variation, the random blur 1808 intensively augments the colors by randomly shifting the global mean and channel means of RGB channels as well as adjusting hue, saturation, and contrast.

Similar data may be used for validation. However, during validation, the fixed augmentation may be applied on the whole image instead of applying random augmentation on the true HQ patches 1806. For color normalization, H&E channels may be separated by deconvoluting the HG images 1802 with sparse nonnegative matrix factorization, and HG images 1802 may be restored with standard H&E staining matrix.

In one implementation, and adaptive learning rate optimization algorithm with a small learning rate (e.g., 1×10−4) may be used to update model weights of the image restoration architecture 1800. The weights of the encoder-decoder generator 1812 and the pixel discriminator 1816 are updated alternatively in each iteration. In one example, an entirety of the training and validation process may run up to approximately 400 epochs on two 16 GB processors in approximately 6 hrs. In one implementation, the encoder-decoder generator 1812 generates fake HQ image patches 1814. The generated fake HQ image patches 1814 are compared with the true HQ image patches 1806 by includes Peak Signal-to-Noise Ratio (PSNR) and Structural Similarity Index (SSIM) scores on the validation set 116, which may be a subset of the training set 114.

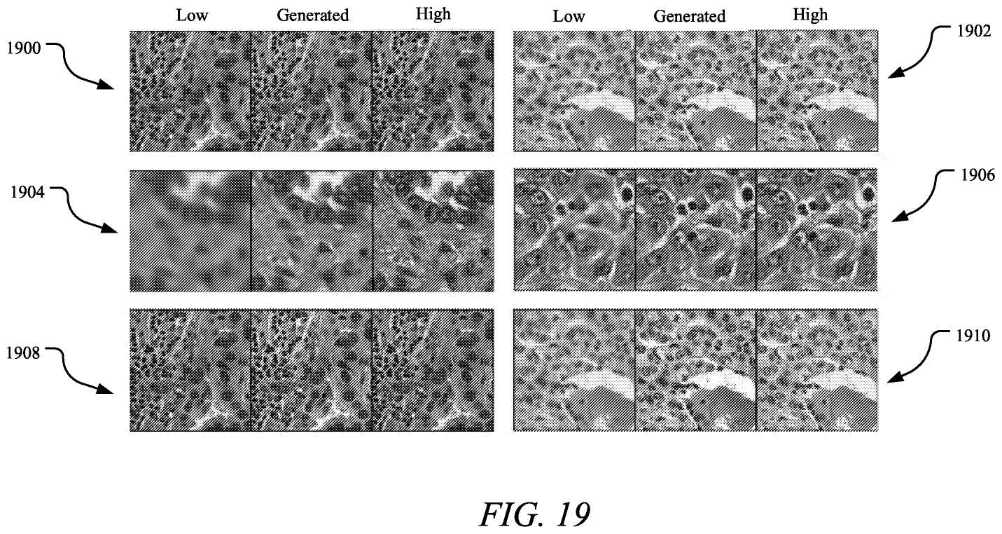

During the inference, the encoder-decoder generator 1812 alone may be used to enhance the quality of a given image. The image restoration architecture 1800 contains only 12.5 m parameters and can inference a batch of images in approximately 0.145 s or less. The SSIM and PSNR between true HG patch 1806 and the generated images (e.g., the LQ image patch 1810 and the fake HQ image patch 1814) may be computed without color normalization and other algorithms to demonstrate performance of the image restoration architecture 1800. In connection with image deblurring, images generated by the image restoration architecture 1800 achieve a higher SSIM and PSNR in both validation and testing compared to other algorithms. In connection with enhancing resolution, the image restoration architecture 1800 has similar superior performance. The deblur effect and enhance resolution effect of the image restoration architecture 1800 of the image restoration system 122 is visualized in FIG. 19.

The example visualizations in FIG. 19, include: a first series 1900 of images comprising and 10× patch enhanced to 40× through 4× super resolution; a second series 1902 of images showing deblurring of a global Gaussian blurred image; a third series 1904 of images showing restoration of a severe median blurred image; a fourth series 1906 of images showing restoration of a local blurred region (corresponding to the red triangle); a fifth series 1908 of images illustrating enhanced resolution and color normalization; and a sixth series 1910 of images shows deblurring of a global Gaussian blurred image and color normalization. Each of the series 1900-1910 contains three channels: a low quality patch, an enhanced result, and a high quality ground truth. The series 1900-1906 are enhanced/restored patches and the series 1908-1910 are synthesized images. The series 1900-1910 indicate that the image restoration architecture 1800 integrates successfully restored blurred regions and enhanced resolution using one algorithm.

As discussed herein, the image restoration architecture 1800 includes a color normalization option to normalize different staining variations to standard H&E staining. Comparing the series 1900 and 1902 with the series 1908 and 1910, the colorization differences between the enhancement without and with color normalization are visualized. The normalized patches visually share the same quality as the standard version but with more realistic purplish H&E style instead of the original bluish frozen style. Due to the color differences, indicators, such as SSIM and PSNR, may not suitable to directly measure the quality of colorized images.

The optimized images generated by the image restoration system 122 increase performance of the system 100 in providing image classification to correctly classify a given region/patch as normal tissue, cancer region, or white region, as described herein. For example, while other algorithms may be severely penalized due to blurred images during training and validation, the optimized images generated by the image restoration system 122 significantly increase classification accuracy. When looking into the sensitivity and specificity for tumor patches against normal patches, blurring effects may lead to large amounts of tumor tissue patches being misclassified into normal regions (low sensitivity/TPR) in other algorithms, while the image restoration system 122 successfully recovers the abnormally low sensitivity to standard level.

Reduced resolution of patches may also lead to low accuracy and low sensitivity of classification in other algorithms. For example, some algorithms with reduced resolution patches may see classification accuracy decreased around 10% on 10× patches and 1-3% on 20× patches. On the other hand, with the optimized images generated through the image restoration system 122, the system 100 achieves the same accuracy as high resolution patches at 20× patches, and for 10× patches enhanced by the image restoration system 122, the accuracy is significantly improved (e.g., by 8% on both validation and testing) and almost reaches the same level as the original high resolution patches. Similar to blurring images, low sensitivity is observed on running the classifier on low resolution patches and can be recovered after applying the image restoration system 122 to enhance the resolution. As such, the image restoration system 122 outperforms other algorithms in enhancement, deblurring, and/or the like, thereby increasing classification accuracy.

The optimized images generated through the image restoration system 124 may further optimize segmentation performance by the system 100, including increasing accuracy of detection and classification of a nuclei and segmentation of its region. Nuclei detection coverage and classification accuracy of a Mask R-CNN model and similar algorithms may be significantly decreased when using blurred validation and testing images. When these blurred images are restored using the image restoration system 122, the accuracy of a Mask R-CNN model is significantly increased, with over 97% of cells accurately classified compared to a benchmark. Additionally, the detection coverage rate during validation is much higher than other algorithms, demonstrating the image restoration system 122 restores blurred regions/patches with significant improvement, and the images restored by the mage restoration system 122 are optimized for Mask R-CNN models and similar algorithms.

As described herein, a Mask R-CNN model may have difficulty in detecting small, crowded objects, such as nuclei. To address these challenges, the system 100 may enhance low resolution patches to high resolution (e.g., 40×) patches and then run the pretrained high resolution Mask R-CNN model on the enhanced patches. For 10× patches, the image restoration system 122 outperforms other methods with around 3% higher coverage with the same level of classification accuracy. For 20× patches, the image restoration system 122 similar outperforms other methods (e.g., loss of 1% on detection coverage). As such, the image restoration system 122 is uniquely positioned to enhance image resolution and provide a feasible way to apply a high resolution instance segmentation algorithm like Mask R-CNN to low resolution images.

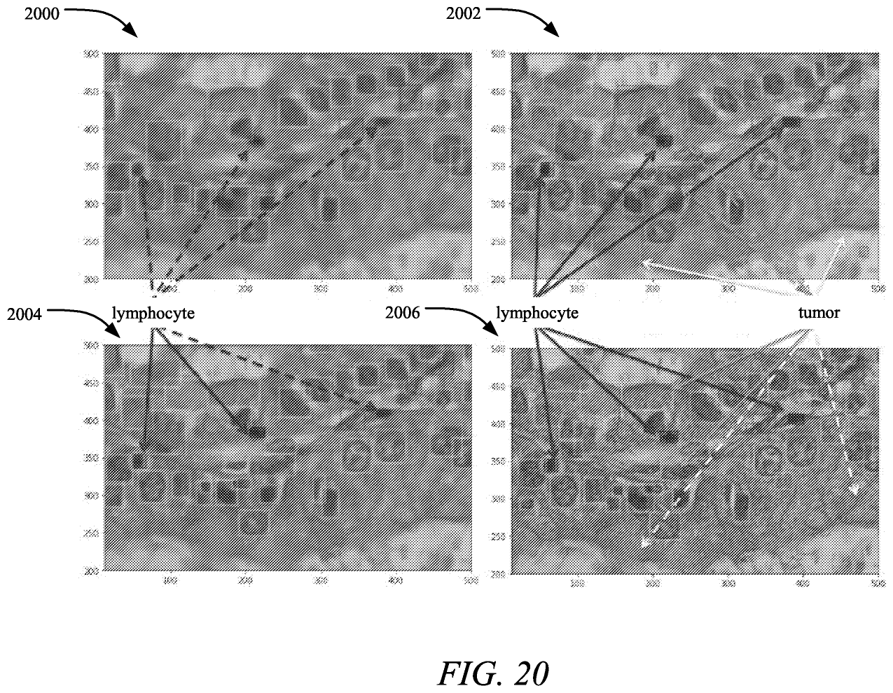

Turning to FIG. 20, it will be appreciated that the image restoration system 122 may be used to facilitate slide collection. For example, the image restoration system 122 may be used to enhance quality and normalize colorization on a low-end scanner. In the example of FIG. 20, for clarity in the original image, only a bounding box is shown instead of a mask of successfully detected nuclei. In the example of FIG. 20, green represents tumor nuclei; red represents stroma nuclei; blue represents lymphocyte nuclei; and pink represents blood cells. A low quality image 2000 is obtained from a scanner, and an optimized image with color normalization 2002 is generated using the image restoration system 122. Comparing the images 2000-2002 to an optimized image without color normalization 2004 generated by the image restoration system 122 and a high quality image 2006 obtained from a scanner, the benefits of the image restoration system 122 are apparent. After enhancing resolution, missing lymphocytes (shown in blue) are successfully recovered by a Mask R-CNN model. After further normalizing the colorization, an extra lymphocyte and 2 tumor nuclei are observed.

An automatic high quality scanner is generally cost prohibitive for many facilities. Moreover, scanning large batch of slides under high resolution (40×) while ensuring quality time consuming and cumbersome. Also, reanalysis of old slides with abnormal colorization is not always feasible with many systems. On the other hand, the image restoration system 122 effectively addresses these issues. FIG. 20 compares the nuclei detection results of the low quality image 2000 and the high quality image 2006 from the same scanner. In this example, the low quality image 2000 is taken under 10× magnitudes with an improper exposure and focal distance, while the high quality image 2006 was taken under 40× magnitudes with precise focal distance. In the optimized image without color normalization 2004, the Mask R-CNN successfully detected three lymphocytes (blue bounding box) that are missing in the low quality image 2000. With the optimized image with color normalization 2002 more nuclei are recovered. As low resolution slides take less storage and scanning slides under 10× magnitude is much faster than 40× magnitude, the image restoration system 122 efficiently scans and stores large amount of slides under low resolution with near 40× quality. The color normalization of the image restoration system 122 further adopts slides with abnormal staining in data collection. The quality and speed enhancement permits a loosening of the criteria of image quality control and thus enlarges available data size for further analysis.

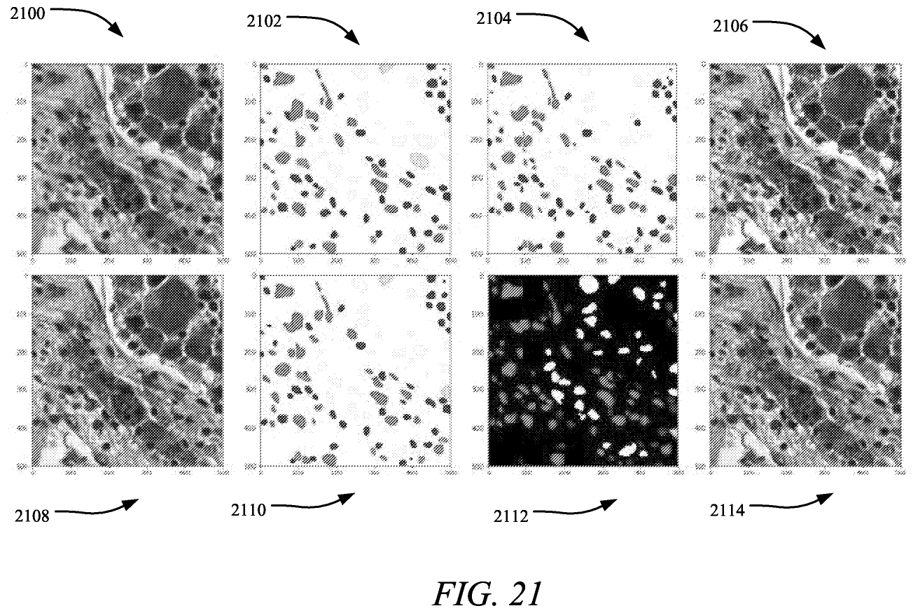

Referring to FIG. 21, the benefits of color normalization by the image restoration system 122 are illustrated. As described herein, staining normalization can reduce the stability of some models if certain color patterns are unobserved during training. However, normalizing staining variation may create misleading information if not handled correctly. FIG. 21 shows a low quality and Mask R-CNN pair 2100, a color normalized optimized image and Mask R-CNN pair 2102, an optimized image and Mask R-CNN pair 2104, and a high quality image and true mask pair 2106.

As shown in FIG. 21, extra lymphocytes and tumor nuclei are observed by the Mask R-CNN using the color normalization. The image restoration system 122 changes a brownish macrophage region into pink through color normalization. While a fine-tuned Mask R-CNN model with intensive color augmentation can still correctly classify macrophage, in some cases, some macrophages are missing and misclassified into other categories that the system 100 resolves. When training or transferring learning a new model, the image restoration system 122 enhances the image quality, and an intensive color augmentation may be applied to increase the model robustness against different colorization style. During the inference, the image restoration system 122 without color normalization may be used if the model is robust during the training. Otherwise, color normalization may be applied if the model is not robust to color variation and all staining patterns have been observed.

Generally, the image restoration system 122 provides an advanced image enhance tool for pathological image analysis. The algorithm of the image restoration architecture 1800 of the image restoration system 122 can recover blurred regions and low resolution images by restoring fine details learned from high quality images. As demonstrated herein, the image restoration system 122 performs better than existing restoration tools for deblurring and provides benefits over state-of-the-art models for enhancing resolution task. The image restoration system 122 provides an efficient way to obtain high-quality pathological images from low-quality scanners and repair the blurred region. The improved images can facilitate downstream imaging analysis and provides a feasible way to cover the limitations of data augmentation and model architectures. This will release the burden of complicated model architecture design and time consuming transfer learning. Furthermore, the image restoration system 122 includes an optional integrated color normalization feature to reduce the staining differences between slides. The system 100 utilizes color normalization with caution to avoid accidentally removing unique features. A merge style-transfer GAN may be merged into the image restoration system 122 to further increase the model capability on frozen slides, fluorescence image, and/or the like.

As described herein, EGFR TKIs are effective for many patients with lung cancer carrying sensitizing EGFR mutations. However, not all patients are responsive to EGFR TKIs, even among those harboring sensitizing EGFR mutations. As such, the system 100 may be used to quantify the cellular interaction features in the TME using routine pathological biopsy images. Using these features, the system 100 generates the prognostic model 108 for the response to EGFR TKI therapy in patients with lung ADC and EGFR-sensitizing mutations. The novo mechanisms of EGFR TKI resistance may further be evaluated using the system 100.

In evaluating the system 100, H&E stained pathology images for patients with EGFR-mutated lung ADC were collected from a first source (n=150), (n=122), and a second source (n=53), respectively. For patients treated with EGFR TKIs in the validation set 116, predicted responders had prolonged survival outcomes compared with predicted non-responders (pv=0.024). Furthermore, patients treated with EGFR TKIs had prolonged survival outcomes in the predicted responders (without versus with EGFR TKI (HR=9.05, pv=0.00005) but not in the non-responders (HR=1.26, pv=0.70).

The system 100 may be used to investigate the genomic profile associated with the image features that are predictive of EGFR TKI response. The tumor-tumor interaction from the tissue images is positively correlated with EGFR TKI response, while the tumor-stroma interaction was negatively correlated with EGFR TKI response. Moreover, tumor-stroma interaction is correlated with higher activation of the HGF/MET-mediated PI3K/AKT signaling pathway, indicating fibroblast-involved resistance to EGFR TKI treatment.

TKIs of the EGFR have shown promising survival improvements in treating patients with lung cancer as first line therapy. Erlotinib, one of the EGFR TKIs, was also the first globally approved targeted therapy for locally advanced or metastatic non-small cell lung cancer. Multiple studies have reported response rates of 60% to 80% with EGFR TKI therapy in patients with NSCLC carrying sensitizing EGFR mutations. Therefore, it is clinically important to prospectively identify the patients most likely to respond to EGFR TKIs as well as those less likely to demonstrate robust response.

As a routine clinical procedure, H&E-stained pathology tissue slides provide detailed tumor morphological characterization at high resolution. There is a relationship between pathological phenotype and targeted therapy response. For example, dominant papillary subtype is predictive for EGFR TKI sensitivity in patients with lung adenocarcinoma (ADC), bronchioloalveolar pathologic subtype (which may represent several different growth patterns today) is associated with EGFR TKI efficacy in patients with NSCLC; and there are overlapping characteristics between the bronchioloalveolar subtype and terminal-respiratory-unit (TRU) type lung ADC, for which EGFR mutation is specific. However, there was a lack of objective quantification, independent validation, and biological characterization of histopathological features and their predictive value in the context of response to EGFR TKI. With the development of whole slide image scanning techniques and deep-learning based image analysis methods of the system 100, the computational analysis of pathology images by the system 100 has tremendous potential to assist pathologists with cancer diagnosis and prognosis.

The system 100 uses the histology-based digital staining system 102 of the instance detection deep learning method, to identify and classify cell types in standard H&E-stained pathology images. The TME that was characterized based on the spatial distribution and organization of these cells has been associated with survival and genomic features of lung ADC patients and was further harnessed to develop a pathology image-based prediction model of the prognostic model 108 for responses to EGFR TKIs in patients with EGFR-mutant lung ADC. The model was further validated in an independent cohort. Gene expression analysis was used in a third independent cohort to examine potential mechanisms of resistance suggested by the risk prediction model of the prognostic model 108.

In one example, the training set 114 includes 168 patients with sensitizing EGFR mutation treated with EGFR TKI therapy and have both response and pathology imaging data. Patients whose pathology images only contained blood (n=12) or stroma (n=6) tissue without tumor cells according to the histology-based digital staining analysis results by the histology-based digital staining system 102 were excluded from the analysis. Therefore, H&E-stained pre-EGFR TKI treatment pathology slides (n=178, 22 patients with ≥2 slides available) and the corresponding clinical information for 150 patients with EGFR-mutant lung ADC who received EGFR TKI treatment were used as the training set 114 to develop a survival model distinguishing patients who responded to EGFR TKI treatment or not.

In this example, the validation set 116 was derived from 127 patients had a EGFR mutation detected and had both clinical information and pathology image data available. Patients whose pathology images only contained blood (n=3) or stroma (n=2) tissue were excluded from the analysis. Therefore, H&E-stained pathology slides (n=132, 7 patients with ≥2 slides available) and the corresponding clinical information for 122 patients with EGFR-mutant lung ADC were used as the independent validation set. Of these, 88 patients carried sensitizing EGFR mutation and treated with EGFR TKI, while the remaining 34 patients did not receive an EGFR TKI treatment (16 of whom carried sensitizing EGFR mutation and the resting 18 patients carried other EGFR mutation). Although patients with sensitizing EGFR mutation typically receive targeted therapy, a variety of factors including rapid clinical decline after enrollment and loss of follow-up may prevent that therapeutic intervention. However, the reduced survival of untreated patients was not clearly attributable to early death after enrollment. Clinical characteristics of the sources in the validation set 116 were similar, though one cohort had a lower proportion of women and enrolled patients tended to have more advanced stage disease relative to the other cohort. Patient characteristics of training, validation and analysis are shown in Table 3.

TABLE 3

| | | | | | Genomic | | P |
|---|---|---|---|---|---|---|---|
| | Training | | Validation | | analysis | | value |
| # EGFR | 150 | | 122 | | 53 | | |
| mutated | | | | | | | |
| # EGFR TKI | 150 | | 88 | | 2 yes, 20 no, | | |
| treated | | | | | 31 unknown | | |
| # Biopsy slides | 178 | | 132 | | 68 | | |
| Age (year) | 61.5 ± 10.5 | | 62.4 ± 10.3 | | 65.2 ± 8.9 | | 0.076 |
| Gender (%) | | | | | | | 0.10 |
| Male | 33 | (22%) | 40 | (33%) | 17 | (32%) | |
| Female | 117 | (78%) | 82 | (67%) | 36 | (68%) | |
| Smoking | | | | | | | <0.001 |
| status (%) | | | | | | | |
| Current | 1 | (1%) | 3 | (2%) | 9 | (17%) | |
| Former | 56 | (37%) | 54 | (45%) | 22 | (42%) | |
| Never | 93 | (62%) | 64 | (53%) | 22 | (42%) | |
| Surgery | | | | | | | <0.001 |
| received? (%) | | | | | | | |
| No | 87 | (58%) | 79 | (65%) | 0 | (0%) | |
| Yes | 63 | (42%) | 41 | (34%) | 53 | (100%) | |
| Unknown | 0 | (0%) | 2 | (1%) | 0 | (0%) | |
| Stage at | | | | | | | <0.001 |
| diagnosis (%) | | | | | | | |
| I | 13 | (9%) | 5 | (4%) | 27 | (51%) | |
| II | 8 | (5%) | 3 | (2%) | 15 | (28%) | |
| III | 23 | (15%) | 10 | (8%) | 6 | (11%) | |
| IV | 104 | (69%) | 101 | (83%) | 5 | (9%) | |
| Unknown | 2 | (1%) | 3 | (2%) | 0 | (0%) | |

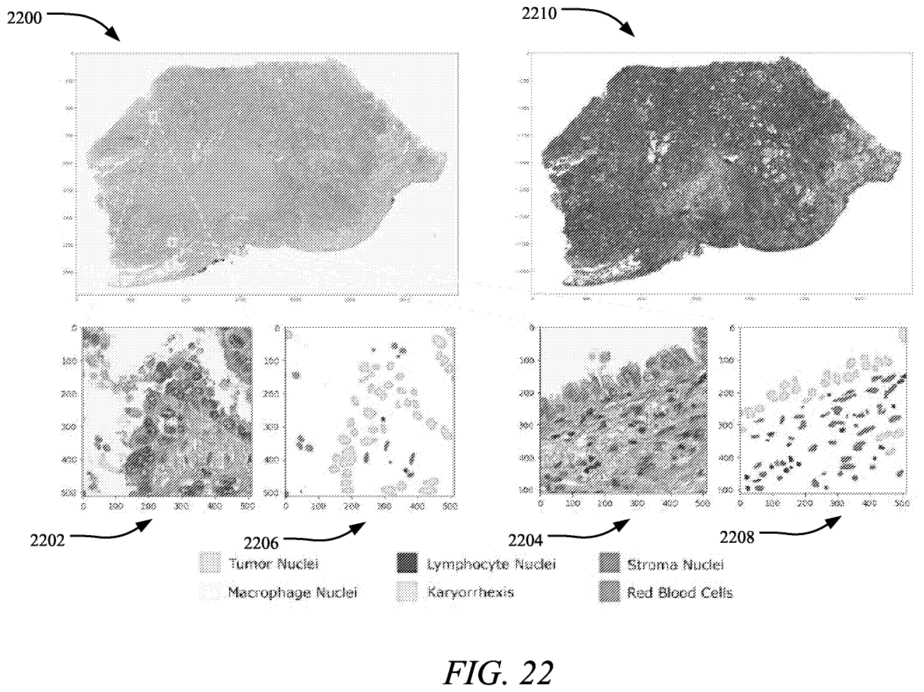

The histology-based digital staining system 102 includes an instance segmentation deep neural network trained for lung cancer pathology, to identify different cell types, including, without limitation: tumor cells, stromal cells, lymphocytes, red blood cells, macrophages, and karyorrhexis from H&E-stained images. The histology-based digital staining system 102 may be applied to whole pathology slides under 40× magnification as illustrated in FIG. 22. The cell type may be identified and centroid location of each identified cell nuclei may be determined for the purpose of characterizing cell-cell interactions. Histology-based digital pathology images from the datasets were a mixture of slides captured at 20× and 40×; the images captured at 20× were resized to 40× using a fine-tuned super-resolution generative adversarial network 122, and then the histology-based digital staining system 102 was applied. From an original image 2200, a first ROI 2202 and a second ROI 2204 are identified and staining images 2206-2208 are generated. A whole slide image histology-based digital staining image 2210 is provided.

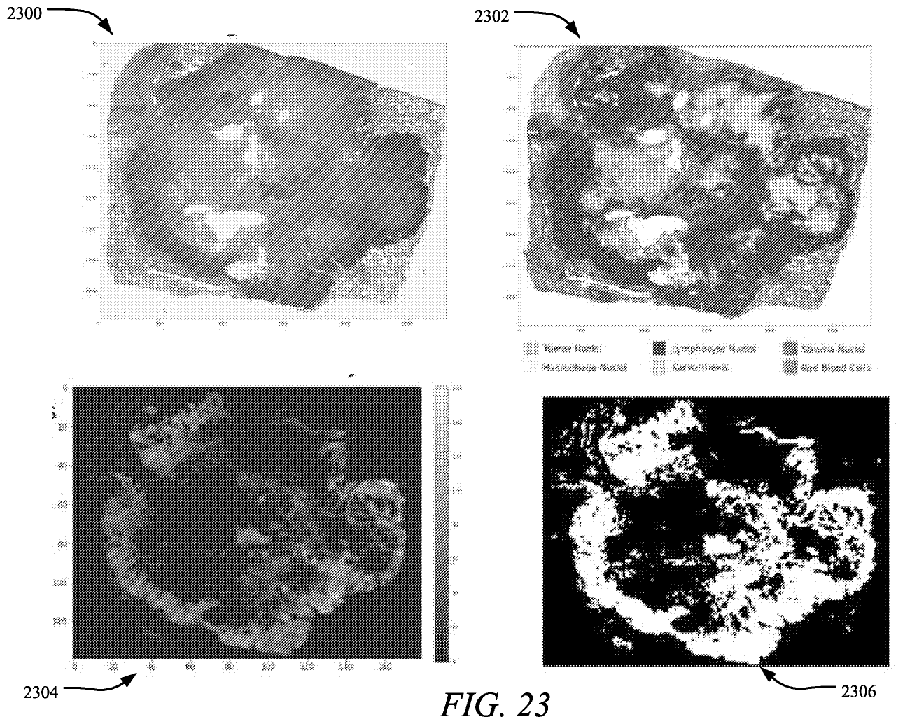

Referring to FIG. 23, image regions with tumor nuclei density >10 per 500×500 pixel image were classified as tumors. Up to one hundred 1024×1024 pixel image patches (on average, 83.7 and 82.6 patches were selected per patient for a first dataset and a second dataset of the first source, respectively), with spatial resolution of 0.25 μm per pixel, were randomly selected from tumor regions of each patient. The cell density of each nucleus type and interactions between tumor cells and their neighbors were extracted for each image. In the example where six nucleus categories were identified by the histology-based digital staining system 102, the density of each type of nucleus was calculated (yielding six image features). To quantify the interactions between tumor cells and their neighbors, the cell organization in each image patch was characterized using a Delaunay triangle graph as previously described. Cellular interactions were defined as:

$$tumor ‐ X interaction = \frac{\#tumor ‐ X connection}{Σ k in all cell types \#tumor ‐ k connection}$$

where X and k refers to one of the following nucleus types: tumor, stroma, lymphocyte, red blood cell, macrophage, and karyorrhexis. For example, the equation quantified tumor-stroma interaction as the ratio of tumor cell-stroma cell connection numbers to the number of connections between tumor cells and all their neighbors. The tumor-stroma interaction was a numeric value, ranging from 0 to 1 and denoted the % interaction with stroma cells. The image features were averaged across all image patches extracted from the tumor region. Average values were calculated for patients with multiple slides (yielding another 6 image features). In total, twelve image features were extracted for each patient.

As shown in FIG. 23, an original slide 2300 is input into the histology-based digital staining system 102 to generate a histology-based digital staining output 2302 from which tumor cell density plot 2304 is generated. Select regions with a density of 10 or less may be identified to generate a tumor region detection plot 2306. Each pixel in the tumor cell density plot 2304 corresponds to a 500×500 pixel image patch under 40× magnification. The regions with tumor nuclei (stained in green in the histology-based digital staining output 2302) density of 10 or less per 500×500 pixel image patch were classified as tumors (white region in the tumor region detection plot 2306).

As described herein OS is defined as the date of diagnosis of metastatic disease till death or last contact. OS was used as the response to EGFR TKI treatment for survival analyses. A Cox Proportional Hazard (CoxPH) model for OS was developed in the first source dataset to correlate with response to EGFR TKI treatment. An elastic net penalty was used to avoid overfitting and select the final 2 features from the 12 input image features.

The response prediction model of the prognostic model 108 generated by the system 100 calculates the risk score 112 for one patient by summing the products between features and corresponding coefficients. The coefficient for tumor-tumor interaction may be, for example, −1.082 and the coefficient for tumor-stroma interaction 0.407. A higher risk score indicates worse response to TKI treatment. To validate the response prediction model of the prognostic model 108, risk scores were calculated for the second cohort, and a median cut-point divided patients into two groups, EGFR TKI responders or non-responders. The survival difference between patients receiving EGFR TKI treatment and patients who did not was estimated using the Kaplan-Meier method and log-rank test in the responders and non-responders. The interaction between the predicted responders and EGFR TKI treatment was evaluated using a multivariate CoxPH model after adjusting for other clinical variables, including age, sex, smoking status, and surgical resection.

To define the biological mechanisms underlying relationships between image features and EGFR TKI treatment response, gene expression data of 53 patients with EGFR mutant lung ADC from the second source were collected and preprocessed. Genes whose mRNA expression levels were 0 in more than 20% of patient samples were removed. The correlations between mRNA expression levels and image-derived cellular interactions were evaluated using Spearman's rank correlations. GSEA was performed for each image feature selected from the EGFR TKI response prediction model of the prognostic model 108. GSEA p-values were adjusted using the Benjamini-Hochberg procedure. Two-sided p-values<0.05 were considered significant.

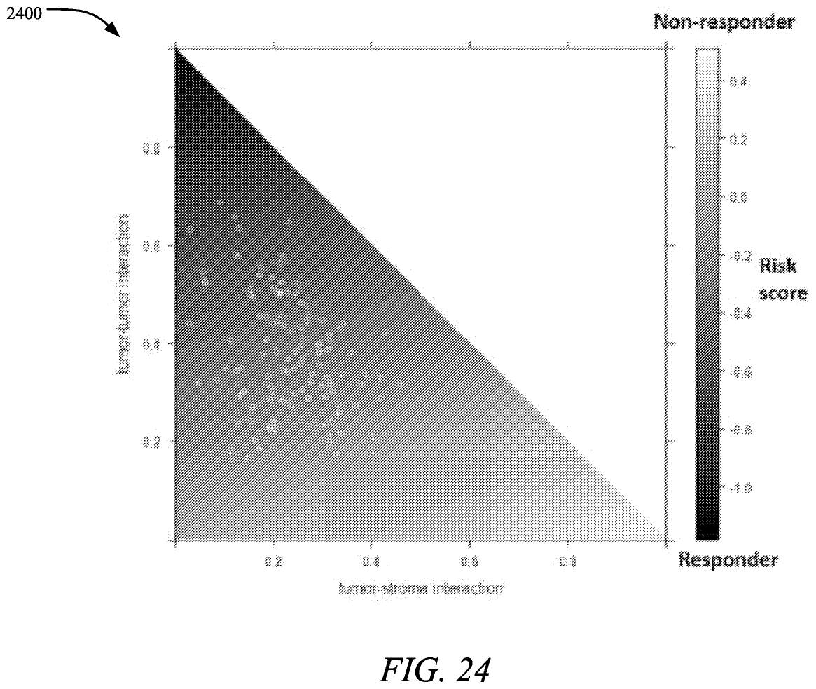

Turning to FIG. 24, in one example, to quantify nuclei composition and interactions between tumor cells and their neighbors in the TME, twelve image features were extracted from the tumor region and used to predict response to EGFR TKI therapy following the pathology image analysis pipeline of the system 100. Two of the twelve image features, tumor-tumor interaction and tumor-stroma interaction, were significantly correlated with response to TKI therapy in the training set 114 (per 10 percent tumor-tumor interaction, HR=0.73, P=0.004; per 10 percent tumor-stroma interaction, HR=1.54, P=0.009) and selected by the CoxPH model. As shown in a plot 2400, tumor-tumor interaction was associated with better EGFR TKI response (lower risk score), while higher tumor-stroma interaction was associated with worse response (higher risk score).

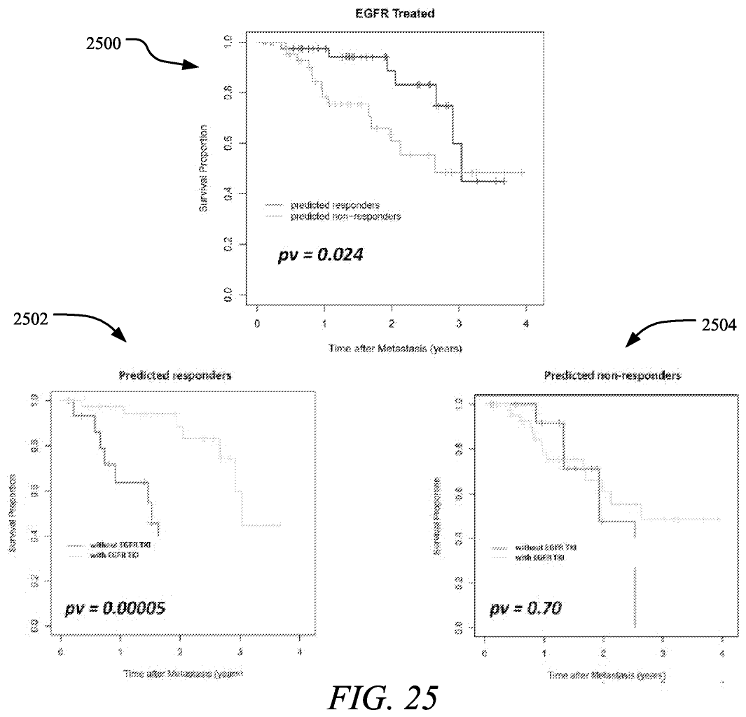

Referring to FIG. 25, a plot 2500 of EGFR treated is shown. The EGFR TKI response prediction performance was validated in the independent dataset. Within the 87 patients who both carried sensitizing EGFR mutation and received EGFR TKI therapy, 42 patients who were predicted as responders showed significantly better OS than the 45 patients who were predicted as non-responders (P=0.024). As can be understood from the plot 2500, survival curves of predicted responders and non-responders in the EGFR TKI treated group of the validation set 116 were plotted with a Kaplan-Meier plot. All treated patients carried sensitizing EGFR mutation. The P-value was estimated with the log-rank test.

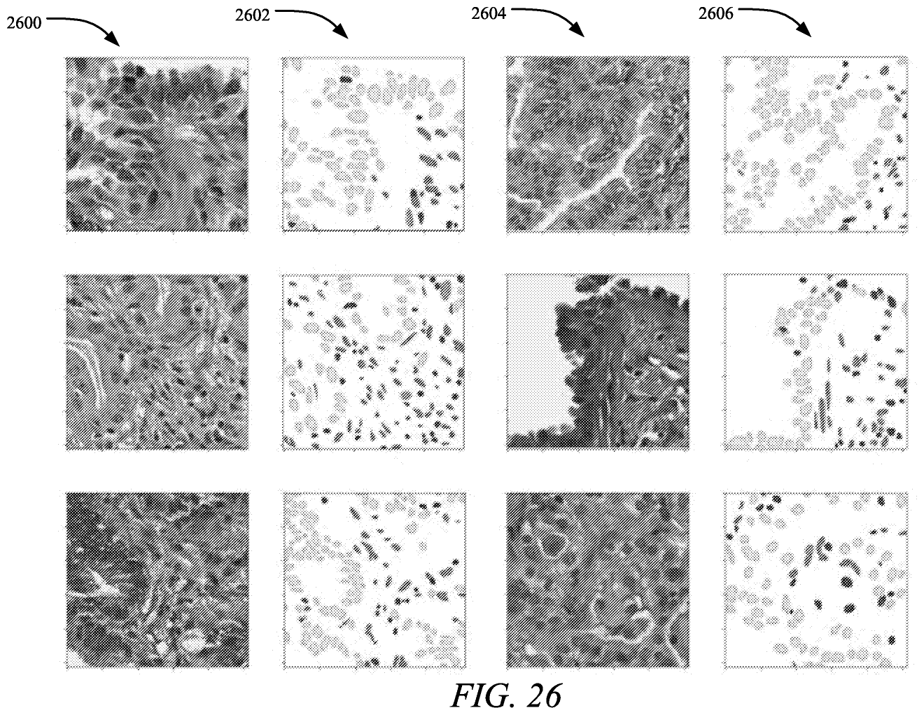

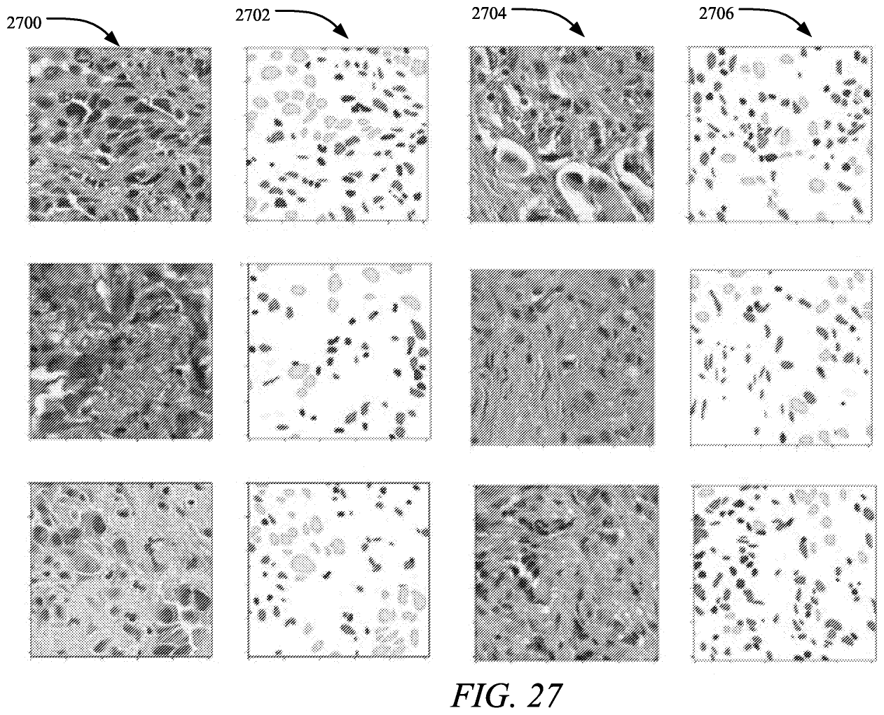

A predicted responders plot 2502 and a predicted non-responders plot 2504 are shown in FIG. 25. Within those predicted responders, sixteen patients who did not receive EGFR TKI therapy showed significantly worse OS than the 42 patients who received EGFR TKI therapy, as shown in the plot 2502 (n=58, P<0.001; HR=9.05, 95% Confidence Interval [CI] 2.57-31.93). In contrast, EGFR TKI treatment did not impact OS within the predicted non-responders as shown in the plot 2504 (n=60 [45 received EGFR TKI therapy and 15 did not], P=0.70; HR=1.26, 95% CI 0.44-3.57). After adjusting for potential clinical confounders, including age, sex, smoking status, and surgery, the interaction between EGFR TKI therapy and predicted responders was still significant as can be understood from Table 3 (P=0.046). The predictive value was still observed after further stratifying patient groups with EGFR mutation type as can be understood from FIG. 26-27. From the pathological aspect, images from patients in the responders group appeared more proliferative, while more tumor-stroma interfaces were observed in the non-responders, consistent with the model coefficients illustrated in the plot 2400. FIG. 26 illustrates first original images 2600, first histology-based digital staining outputs 2602, second original images 2604, and second histology-based digital staining outputs 2606 of predicted TKI responders. FIG. 27 illustrates first original images 2700, first histology-based digital staining outputs 2702, second original images 2704, and second histology-based digital staining outputs 2706 of non-responders.

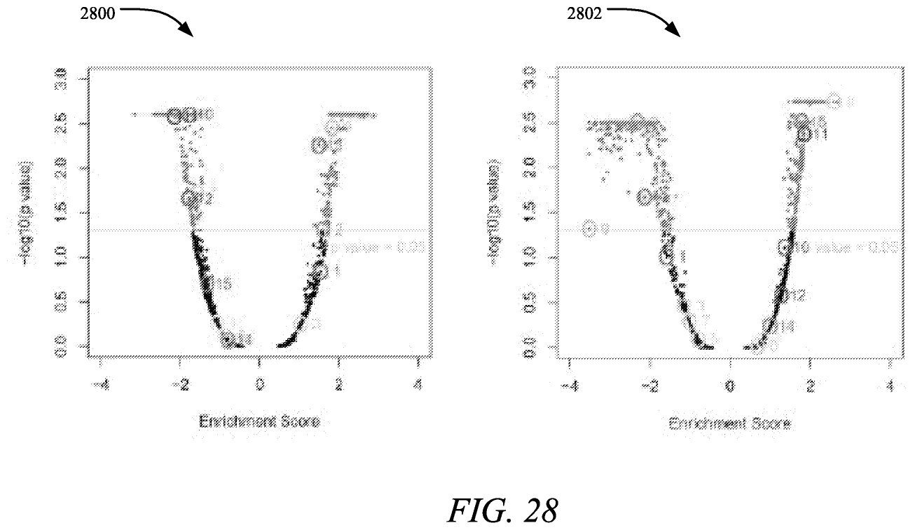

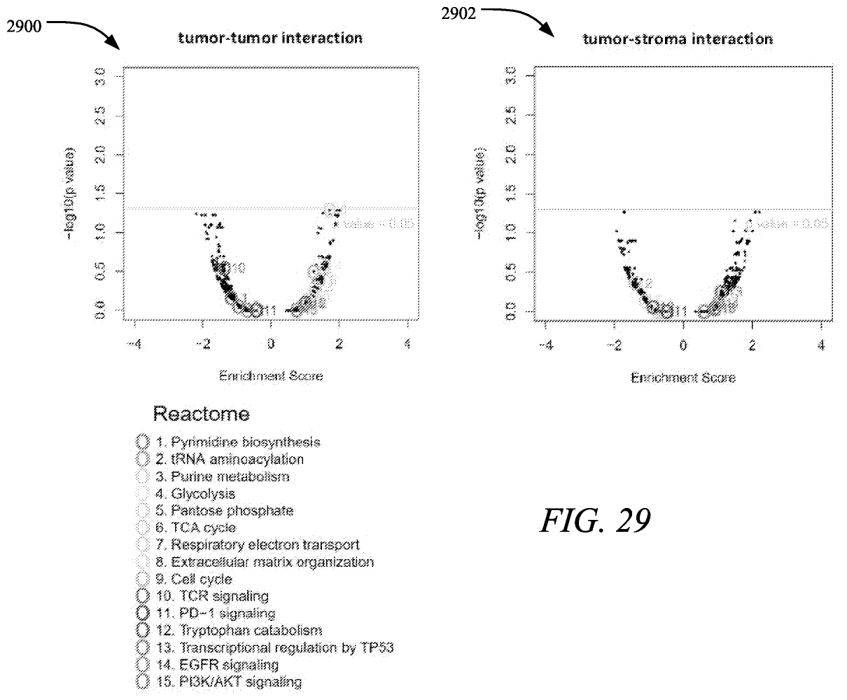

Referring to FIG. 28, to understand the biological mechanisms underlying the predictive value of image-based tumor-tumor interactions and tumor-stroma interactions for EGFR TKI therapy response in patients with EGFR mutations, the system 100 performs association analysis between image features and mRNA expression for patients with EGFR mutation. GSEA analysis identified multiple biological pathways whose mRNA expression profiles significantly correlated with tumor-tumor interaction as shown in the plot 2800. The results showed that transcriptional activation of the cell cycle pathway and transcription regulation pathway by TP53 positively correlated with tumor-tumor interaction. In contrast, transcriptional activation of extracellular organization, the PD-1 signaling pathway, and the PI3K/AKT signaling in cancer pathway positively correlated with tumor-stroma interaction, as shown in FIG. 2802. The EGFR signaling in cancer reactome did not correlate with either tumor-tumor interaction or tumor-stroma interaction, showing that EGFR expression is not a good predictive factor for EGFR TKI response. As a negative control, the patient IDs were randomly shuffled and the analysis repeated, and the positive correlations was no longer observed as shown in FIG. 29, illustrating a tumor-tumor interaction plot 2900 and a tumor-stroma plot 2902.

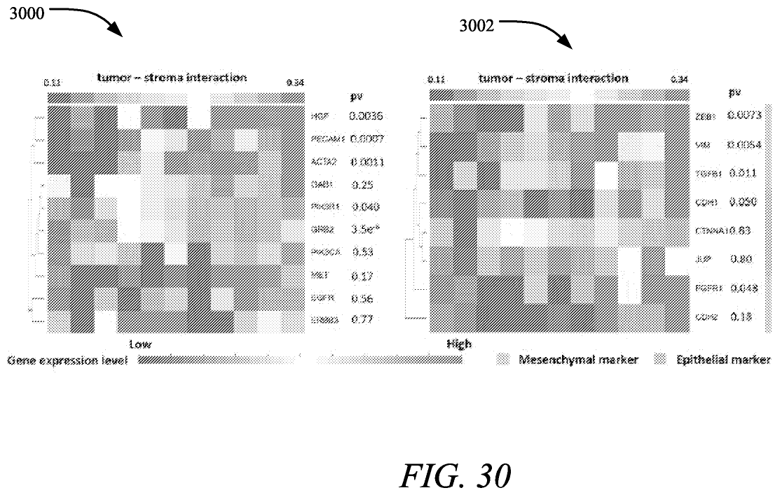

To test the hypothesis that image-derived tumor-stroma interaction could reflect crosstalk between tumor cells and fibroblasts, the system 100 analyzed the relationship between tumor-stroma interaction and the expression of the stromal markers ACTA2 (α-smooth muscle actin [αSMA], a marker for activated fibroblast) and PECAM1 (CD31, a marker for angiogenesis). A strong correlation between tumor-stroma interaction and mRNA expression of ACTA2 and PECAM1 was observed as shown in a tumor-stroma interaction plot 3000 shown in FIG. 30.

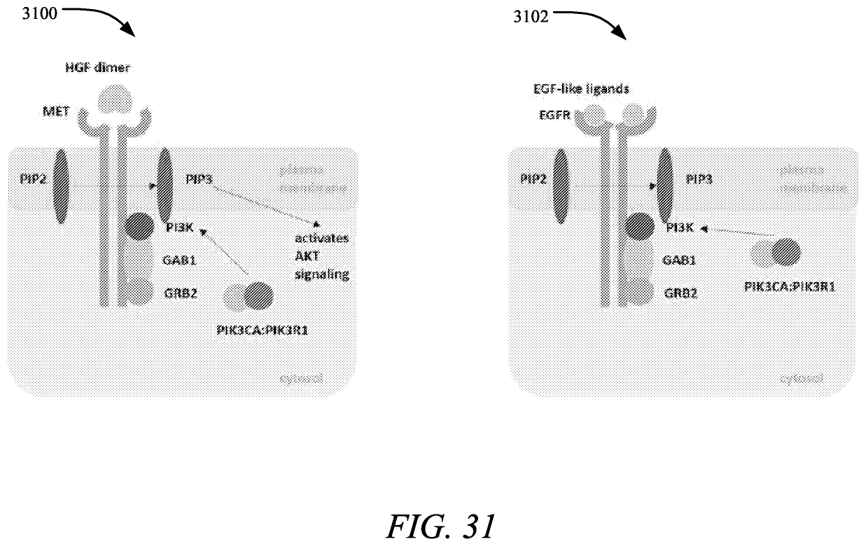

When cocultured with HGF-secreting fibroblasts, EGFR-mutant lung cancer cell lines become resistant to EGFR TKIs. Therefore, we compared expression of genes involved in HGF-mediated PIP3 activation, as shown in the diagram 3100 of FIG. 31, with EGFR-mediated PIP3 activation, as shown in the diagram 3102 of FIG. 31.

As shown in a tumor-stroma plot 3002 in FIG. 30, GAB1, GRB2, PIK3R1, and PIK3CA are involved in both pathways. Tumor-stroma interactions reflected activation of fibroblast cells and that HGF secretion were predominantly attributed to stromal cells. Transcription of HGF significantly correlated with tumor-stroma interaction. PI3K/AKT pathway activation is a consequence of MET activation, consistent with the activated PI3K/AKT pathway in tumors with a high level of tumor-stroma interaction plot 2902. In contrast, no significant correlation between tumor-stroma interactions and EGFR expression was detected. ERBB3, which effectively activated the PI3K/AKT pathway exclusively in EGFR TKI sensitive NSCLC cell lines, did not correlate with tumor-stroma interactions of the tumor-stroma interaction plot 3000.

As described herein, the system 100 provides an image-based model to predict EGFR TKI therapy response in patients with EGFR-mutant metastatic lung ADC. The histology-based digital staining system 102 was used to quantify cell composition and cellular interactions within TME. By fitting OS with image features, a higher tumor-tumor interaction was found to correlate with better EGFR TKI response, while a higher tumor-stroma interaction correlated with worse EGFR TKI response. The predictive value of the image-based model of the system 100 was validated in an independent cohort, in which, the predicted responders showed significantly improved OS after EGFR TKI treatment, while the predicted non-responders did not. The system 100 thus provides a novel predictive model based on quantification of pathology images.

Furthermore, to understand the biological mechanisms of EGFR TKI resistance, either intrinsic or acquired, the genetic and proteomic difference among patients, cell lines, or xenografts may be compared with different sensitivity to EGFR TKIs. The deep-learning-aided quantification strategy of the system 100 enables unbiased analysis to associate phenotypic tumor morphology with underlying biological mechanisms.

Crosstalk between tumor cells and fibroblasts has been investigated in vitro and in vivo as a potential therapeutic target and source of EGFR TKI resistance. The system 100 validates observations regarding the role of fibroblast cells in EGFR TKI resistance. The validity of using tumor-stroma interactions as assessed on H&E slides to represent crosstalk between tumor cells and fibroblasts was supported both genetically and phenotypically. HGF-mediated PIP3 activation can likely bypass EGFR-mediated PIP3 activation, as shown by elevated HGF expression in tumors with higher tumor-stroma interactions. HGF secretion is predominantly attributed to stromal cells, but tumor-derived HGF has also been reported in EGFR TKI-resistant tumor cells. While HGF may activate MET in a paracrine or autocrine way, elevated tumor-stroma interactions were associated with unfavorable responses to EGFR TKI in clinical practice.

In addition to EGFR TKI resistance, carcinoma associated fibroblasts (CAF) are also associated with epithelial-mesenchymal transition (EMT). Moreover, EMT may be a predictor for EGFR TKI resistance and a mechanism of acquired TKI resistance. As a predictor of EGFR TKI insensitivity, tumor-stroma interaction also correlated with expression of classic mesenchymal markers, vimentin (VIM), TGFB1, FGFR133, and ZEB1, a driver gene of the EMT process, as shown in the plot 3002. As a comparison, no significant correlation between tumor-stroma interaction and classic epithelial markers, namely E-cadherin (CDH1), alpha-cadherin (CTNNA1), and gamma-catenin (JUP) was observed, as can be understood from the plot 3002. The correlation with transcription of mesenchymal markers may indicate activation of the EMT process for tumors with more tumor-stroma interactions. However, since increased tumor-stroma interactions were indicative of an accumulation of fibroblasts, consistent with the expression data from the TCGA cohort, the correlation may indicate a higher proportion of fibroblasts instead of EMT. Single cell sequencing and immunohistochemical staining using the system 100 addresses this gap.

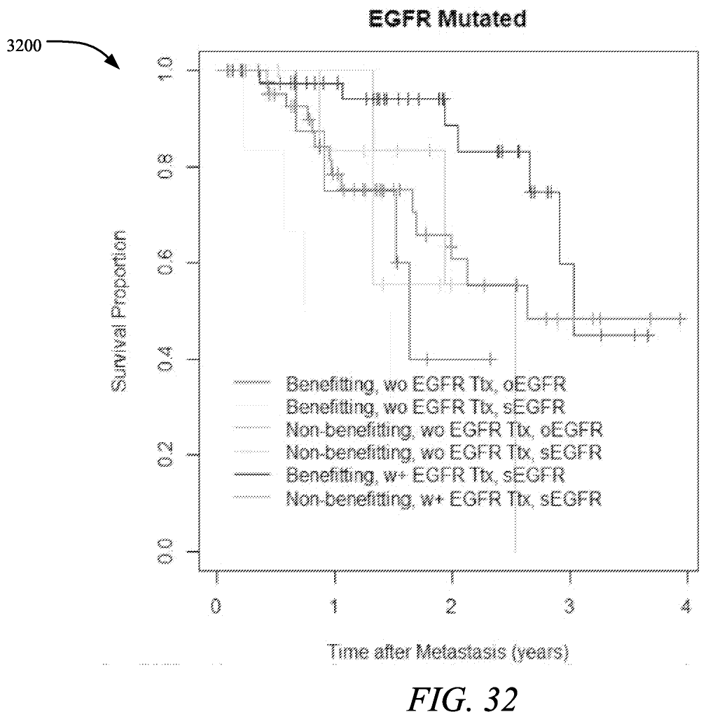

Although AKT activation may be a predictor for sensitivity to EGFR TKIs, the underlying biological mechanism is more complex. Multiple upstream pathways for PI3K/AKT activation have been identified using the system 100, including the EGFR-mediated pathway and the HGF/MET pathway. Sensitivity to EGFR TKI treatment was observed in tumors where EGFR appeared to be the primary mediator of PI3K/AKT pathway activation. However, in patients who do not respond to EGFR TKI therapy, the PI3K/AKT pathway may be activated via bypass signaling through HGF/MET instead of EGFR. Thus, for patients with both sensitizing EGFR mutations and extensive tumor-stroma interactions, additionally targeting HGF/MET may restore the response to EGFR TKIs. Referring to FIG. 32, a survival 3200 in EGFR mutated patients is shown to compare patient groups stratified by predicted responding group, EGFR TKI treated or not, and EGFR mutation type.

The prediction model of the prognostic model 108 generated by the system 100 leverages routine clinical pathology images to identify patients with EGFR-mutant lung ADC who are most likely to respond to EGFR TKI therapy. Further, the system 100 identifies patients who are resistant to EGFR TKI therapy. The results herein demonstrate that combination treatment with EGFR/MET TKI therapies may overcome such resistance. Validation in patients without sensitizing EGFR mutations and in larger datasets could inform clinical trials and translate into better outcome for the overall patient population with lung ADC.

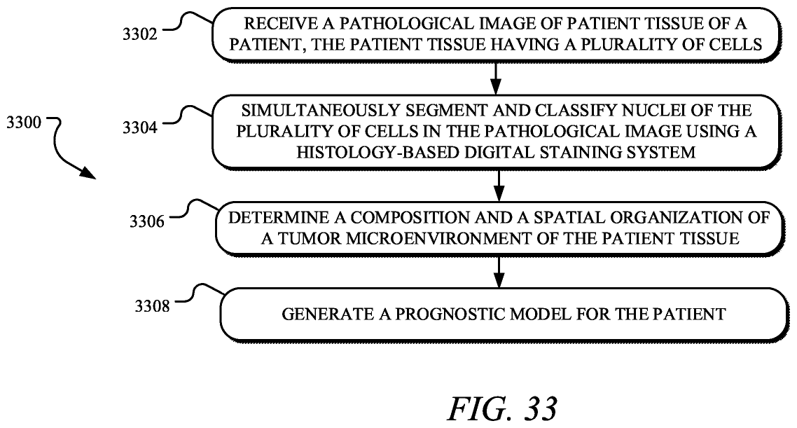

Turning to FIG. 33, example operations 3300 for characterizing patient tissue of a patient are shown. In one implementation, an operation 3302 receives a pathological image of patient tissue of a patient, where the patient tissue includes a plurality of cells. An operation 3304 simultaneously segments and classifies nuclei of the plurality of cells in the pathological image using a histology-based digital staining system. The nuclei of the plurality of cells are segmented according to spatial location and classified according to cell type, thereby generating one or more groups of nuclei. Each of the one or more groups of nuclei has an identified cell type. An operation 3306 determines a composition and a spatial organization of a tumor microenvironment of the patient tissue based on the one or more groups of nuclei. An operation 3308 generates a prognostic model for the patient based on the composition and the spatial organization of the tumor microenvironment.

As detailed herein, the pathology image 104, either a whole image or a patch, may be received at the histology-based digital staining system 102 over a network. Referring to FIG. 34, in one implementation, a user accesses and interacts with the histology-based digital staining system 102 within a network environment 3400 using a user device 3402 to obtain the characterized TME 106 and/or the prognostic model 108, as well as access and interact with other information or services via a network 3404.

The user device 3402 is generally any form of computing device capable of interacting with the network 3404, such as a personal computer, terminal, workstation, desktop computer, portable computer, mobile device, smartphone, tablet, multimedia console, and/or the like. The network 3404 is used by one or more computing or data storage devices (e.g., one or more databases 3406 or other computing units described herein) for implementing the histology-based digital staining system 102 and other services, applications, or modules in the network environment 3400. The pathology images 104, the training pathology images 110, prognostic models 108, characterized TME 106, data, software, and other information utilized by the histology-based digital staining system 102 may be stored in and accessed from the one or more databases 3406.

In one implementation, the network environment 3400 includes at least one server 3408 hosting a website or an application that the user may visit to access the histology-based digital staining system 102 and/or other network components of the network environment 3400. The server 3406 may be a single server, a plurality of servers with each such server being a physical server or a virtual machine, or a collection of both physical servers and virtual machines. In another implementation, a cloud hosts one or more components of the network environment 3400. The user devices 3402, the server 3408, and other resources connected to the network 3404 may access one or more other servers to access to one or more websites, applications, web services interfaces, storage devices, computing devices, or the like that are used for diagnosis, treatment, characterization, analysis, and related services. The server 3408 may also host a search engine that the histology-based digital staining system 102 uses for accessing, searching for, and modifying data, as well as for services, as described herein.

In one implementation, the pathology image is received at the histology-based digital staining system 102 over the network 3404 as the input. Each uploaded input image may be assigned a job ID. The segmentation results will be automatically displayed and the spatial coordinates of each nucleus can be downloaded in a table, as an example. The histology-based digital staining system 102 may be used in connection with TME-related features for various cancer types, such that a function to automatically generate a mask for other cancer types is provided. A newly generated segmentation mask may greatly reduce the manual work of creating the training sets for other cancer types, and thus accelerate the development of applications for pathology image analysis.

Compared with other image segmentation algorithms, the histology-based digital staining system 102 provides several advantages: it segments and classifies nuclei at the same time, while traditional nuclei segmentation algorithms relying on color deconvolution cannot classify cell types; by using extensive color augmentation during the training process, it adapts to different staining conditions, which makes the algorithm more robust and allows us to avoid the time-consuming color normalization steps; compared with traditional statistical approaches, the histology-based digital staining system 102 does not require handcrafted feature extraction and thus be highly parallel and time-saving. For example, with graphical processing unit (GPU) aided computation, processing (classifying or segmenting) a 1000-by-1000-pixel image usually takes less than one second for HD-Staining, much faster than other image segmentation methods. Additionally, compared with other popular semantic image segmentation neural networks that classify each pixel, the histology-based digital staining system 102 is intrinsically an instance segmentation algorithm that detects an object bounding box first and assigns pixels as foreground or background within this bounding box. Overall, the histology-based digital staining system 102 provides a new solution to segmenting closely clustered nuclei in tissue pathology images.

Further, the associations between the extracted TME features and patient prognosis may be understood. Karyorrhexis, a representative of necrosis, has been reported as an aggressive tumor phenotype in lung cancer. Consistently, the density of karyorrhectic cells and numbers of karyorrhexis-karyorrhexis edges were shown as negative prognostic factors. On the other hand, the density of stromal cells and the numbers of stromal cell-stromal cell edges were positive prognostic factors, which is consistent with a recent report on lung ADC patients. These consistencies indicate the validity of the histology-based digital staining system 102 and the potentiality of using cell organization features as novel biomarkers for clinical outcomes.

Gene expression patterns have been widely used to study the underlying biological mechanisms of different tumor types and subtypes. Moreover, genes with abnormal expression could become potential therapeutic targets of cancers. However, traditional transcriptome profiling is usually done in bulk tumor, which contains multiple cell types, such as stromal cells and lymphocytes, in addition to tumor cells. This bulk tumor-based sequencing could blur or diminish the mRNA expression changes arising from a single cell type or from different cell compositions in the TME. Currently, the relationship between the transcription activities of biological pathways and the TME remains unclear. The histology-based digital staining system 102 provides the image-derived TME features, which show correlations with the transcriptional activities of biological pathways. For example, gene expression levels of TCR and PD-1 pathways were positively correlated with the density of lymphocytes detected from tumor tissues. As genes involved in the TCR and PD1 pathways are expressed in immune cells, such correlation illustrates the contribution of lymphocytes to bulk tumor transcriptome profiling and thus validates the accuracy of both image-based nuclei detection and genetic sequencing of bulk tumor. This indicates the image-derived TME features may be used to study or predict immunotherapy response, since several promising cancer immunotherapies rely on activation of tumor-infiltrated immune cells and blocking immune checkpoint pathways. In addition, the gene expression level of extracellular matrix organization pathway is associated with the density of stromal cells in tumor tissues. Since traditional transcriptome sequencing is done in bulk tumor, accurate cell composition derived from pathology images could help to improve the evaluation of gene expression for each individual cell type. Moreover, the correlation between image features and transcriptional patterns of biological pathways hints at the potential usage of image features to study tumor bioprocesses, including cell cycle and metabolism status.

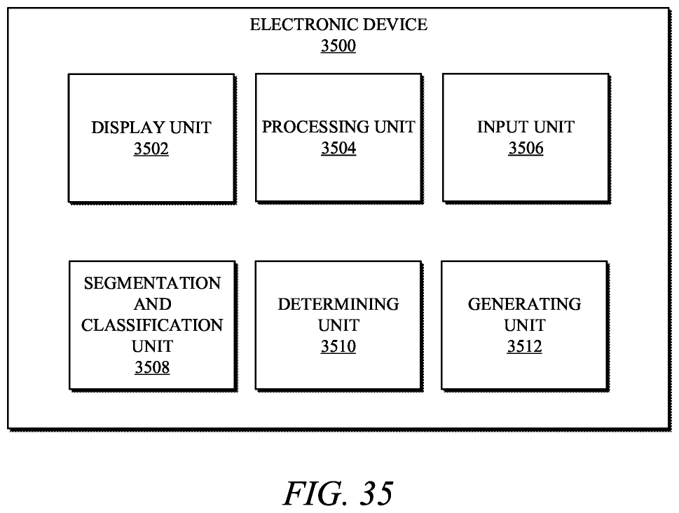

Turning to FIG. 35, an electronic device 3500 including operational units 3502-3512 arranged to perform various operations of the presently disclosed technology is shown. The operational units 3502-3512 of the device 3500 are implemented by hardware or a combination of hardware and software to carry out the principles of the present disclosure. It will be understood by persons of skill in the art that the operational units 3502-3512 described in FIG. 35 may be combined or separated into sub-blocks to implement the principles of the present disclosure. Therefore, the description herein supports any possible combination or separation or further definition of the operational units 3502-3512.

In one implementation, the electronic device 3500 includes a display unit 3502 configured to display information, such as a graphical user interface, and a processing unit 3504 in communication with the display unit 3502 and an input unit 3506 configured to receive data from one or more input devices or systems. Various operations described herein may be implemented by the processing unit 3504 using data received by the input unit 3506 to output information for display using the display unit 3502.

Additionally, in one implementation, the electronic device 3500 includes units implementing the operations described with respect to FIG. 34. For example, the operation 3504 may be implemented by a segmentation and classification unit 3508, the operation 3506 may be performed by a determining unit 3510, and the operation 3508 may be performed by a generating unit 3512.

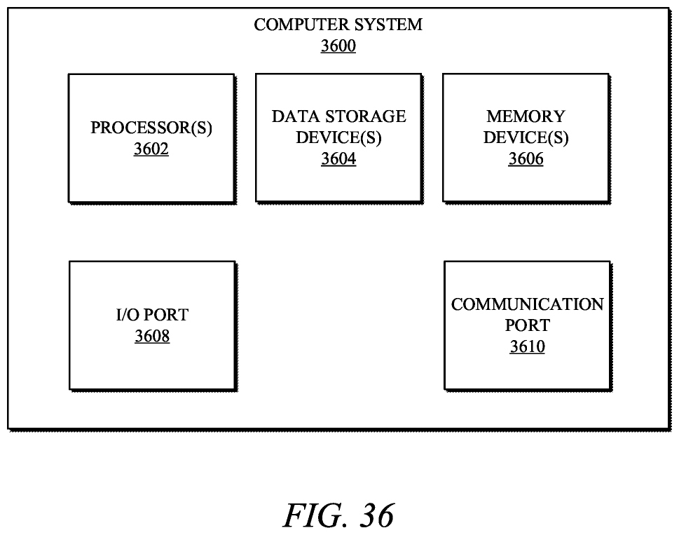

Referring to FIG. 36, a detailed description of an example computing system 3600 having one or more computing units that may implement various systems and methods discussed herein is provided. The computing system 3600 may be applicable to the histology-based digital staining system 102, the user device 3602, the server 3608, and other computing or network devices. It will be appreciated that specific implementations of these devices may be of differing possible specific computing architectures not all of which are specifically discussed herein but will be understood by those of ordinary skill in the art.

The computer system 3600 may be a computing system capable of executing a computer program product to execute a computer process. Data and program files may be input to the computer system 3600, which reads the files and executes the programs therein. Some of the elements of the computer system 3600 are shown in FIG. 36, including one or more hardware processors 3602, one or more data storage devices 3604, one or more memory devices 3606, and/or one or more ports 3608-3610. Additionally, other elements that will be recognized by those skilled in the art may be included in the computing system 3600 but are not explicitly depicted in FIG. 36 or discussed further herein. Various elements of the computer system 3600 may communicate with one another by way of one or more communication buses, point-to-point communication paths, or other communication means not explicitly depicted in FIG. 36.

The processor 3602 may include, for example, a central processing unit (CPU), a microprocessor, a microcontroller, a digital signal processor (DSP), and/or one or more internal levels of cache. There may be one or more processors 3602, such that the processor 3602 comprises a single central-processing unit, or a plurality of processing units capable of executing instructions and performing operations in parallel with each other, commonly referred to as a parallel processing environment.

The computer system 3600 may be a conventional computer, a distributed computer, or any other type of computer, such as one or more external computers made available via a cloud computing architecture. The presently described technology is optionally implemented in software stored on the data stored device(s) 3604, stored on the memory device(s) 3606, and/or communicated via one or more of the ports 3608-3610, thereby transforming the computer system 3600 in FIG. 36 to a special purpose machine for implementing the operations described herein. Examples of the computer system 3600 include personal computers, terminals, workstations, mobile phones, tablets, laptops, personal computers, multimedia consoles, gaming consoles, set top boxes, and the like.

The one or more data storage devices 3604 may include any non-volatile data storage device capable of storing data generated or employed within the computing system 3600, such as computer executable instructions for performing a computer process, which may include instructions of both application programs and an operating system (OS) that manages the various components of the computing system 3600. The data storage devices 3604 may include, without limitation, magnetic disk drives, optical disk drives, solid state drives (SSDs), flash drives, and the like. The data storage devices 3604 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 3606 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 3604 and/or the memory devices 3606, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 3600 includes one or more ports, such as an input/output (I/O) port 3608 and a communication port 3610, for communicating with other computing, network, or vehicle devices. It will be appreciated that the ports 3608-3610 may be combined or separated and that more or fewer ports may be included in the computer system 3600.

The I/O port 3608 may be connected to an I/O device, or other device, by which information is input to or output from the computing system 3600. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 3600 via the I/O port 3608. Similarly, the output devices may convert electrical signals received from computing system 3600 via the I/O port 3608 into signals that may be sensed as output by a human, such as sound, light, and/or touch. The input device may be an alphanumeric input device, including alphanumeric and other keys for communicating information and/or command selections to the processor 3602 via the I/O port 3608. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 3600 via the I/O port 3608. For example, an electrical signal generated within the computing system 3600 may be converted to another type of signal, and/or vice-versa. In one implementation, the environment transducer devices sense characteristics or aspects of an environment local to or remote from the computing device 3600, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 3600, such as, physical movement of some object (e.g., a mechanical actuator), heating or cooling of a substance, adding a chemical substance, and/or the like.

In one implementation, a communication port 3610 is connected to a network by way of which the computer system 3600 may receive network data useful in executing the methods and systems set out herein as well as transmitting information and network configuration changes determined thereby. Stated differently, the communication port 3610 connects the computer system 3600 to one or more communication interface devices configured to transmit and/or receive information between the computing system 3600 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 3610 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 3610 may communicate with an antenna or other link for electromagnetic signal transmission and/or reception.

In an example implementation, pathology images, training images, pathological models, and software and other modules and services may be embodied by instructions stored on the data storage devices 3604 and/or the memory devices 3606 and executed by the processor 3602.

The system set forth in FIG. 36 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

1. One or more non-transitory computer-readable storage media storing computer-executable instructions for performing a computer process on a computing system, the computer process comprising:
receiving a pathological image of patient tissue of a patient, the patient tissue including a plurality of cells;
simultaneously segmenting and classifying nuclei of the plurality of cells in the pathological image using a histology-based digital staining system, the nuclei of the plurality of cells segmented according to spatial location and classified according to cell type, thereby generating one or more groups of nuclei, each of the one or more groups of nuclei having an identified cell type; and
determining a composition and a spatial organization of a tumor microenvironment of the patient tissue based on the one or more groups of nuclei.

2. The one or more non-transitory computer-readable storage media of claim 1, wherein the nuclei of the plurality of cells are segmented and classified by the histology-based digital staining system using a mask regional convolutional neural network.

3. The one or more non-transitory computer-readable storage media of claim 2,
wherein,
the mask regional convolutional neural network is trained using a plurality of training pathological images, and
each of the plurality of training pathological images is manually labeled.

4. The one or more non-transitory computer-readable storage media of claim 1, wherein the patient tissue is at least one of lung tissue, breast tissue, head tissue, or neck tissue.

5. The one or more non-transitory computer-readable storage media of claim 1, wherein the cell type includes at least one of tumor cells, stromal cells, macrophages, red blood cells, lymphocytes, or karyorrhexis.

6. The one or more non-transitory computer-readable storage media of claim 1, wherein the plurality of cells is stained in the image using one or more colors according to the composition and the spatial organization of the tumor microenvironment.

7. The one or more non-transitory computer-readable storage media of claim 1, wherein the composition and the spatial organization of the tumor microenvironment is determined based on image features extracted using connections between centroids of the nuclei of the plurality of cells.

8. The one or more non-transitory computer-readable storage media of claim 7, wherein each of the centroids of the nuclei of the plurality of cells is defined as a vertex on a feature graph and edges between sets of the vertices correspond to the connections for different cell types.

9. The one or more non-transitory computer-readable storage media of claim 1, further comprising:
generating a prognostic model for the patient based on the composition and the spatial organization of the tumor microenvironment.

10. The one or more non-transitory computer-readable storage media of claim 9, wherein the prognostic model includes a risk score.

11. The one or more non-transitory computer-readable storage media of claim 10, further comprising:
assigning the patient to a risk group corresponding to a predicted survival outcome based on the risk score.

12. The one or more non-transitory computer-readable storage media of claim 1, wherein the image is a patch from a larger image.

13. A method for characterizing patient tissue of a patient, the method comprising:
receiving a pathological image of the patient tissue of the patient, the patient tissue including a plurality of cells;
simultaneously segmenting and classifying nuclei of the plurality of cells in the pathological image using a histology-based digital staining system, the nuclei of the plurality of cells segmented according to spatial location and classified according to cell type, thereby generating one or more groups of nuclei, each of the one or more groups of nuclei having an identified cell type;
determining a composition and a spatial organization of a tumor microenvironment of the patient tissue based on the one or more groups of nuclei; and
generating a prognostic model for the patient based on the composition and the spatial organization of the tumor microenvironment.

14. The method of claim 13, wherein a treatment for the patient is optimized based on the prognostic model.

15. The method of claim 13, wherein the composition and the spatial organization of a tumor microenvironment of the patient tissue is determined based on image features extracted from the pathological image using Delaunay triangulation.

16. The method of claim 15, wherein the image features are associated with transcriptional activity of biological pathways.

17. The method of claim 13, wherein the composition of the patient tissue includes one or more different cell types.

18. The method of claim 13, wherein cell type includes at least one of tumor cells, stromal cells, macrophages, lymphocytes, red blood cells, or karyorrhexis.

19. A system for characterizing patient tissue of a patient, the system comprising:
a histology-based digital staining system simultaneously segmenting and classifying nuclei of a plurality of cells in a pathological image of the patient tissue of the patient, the pathological image captured using a tissue slide scanning kit, the nuclei of the plurality of cells segmented according to spatial location and classified according to cell type, thereby generating one or more groups of nuclei, each of the one or more groups of nuclei having an identified cell type, the histology-based digital staining system determining a composition and a spatial organization of a tumor microenvironment of the patient tissue based on the one or more groups of nuclei.

20. The system of claim 19, wherein the pathological image is received from a user device over a network.
